SEQUENCE LISTING (3 sequences)

SEQ ID NO: 1 - protein, 70 aa, Homo sapiens
EICPSFQRVIETLLMDTPSSYEAAMELFSPDQDMREAGAQLKKLVDTLPQKPRESIIKLMEKIAQSSLCN

SEQ ID NO: 2 - protein, 17 aa, Homo sapiens
QNWDTGRERTVNNQSQR

SEQ ID NO: 3 - protein, 21 aa, Homo sapiens
NGTDGASQKTPSSTGPSPVFD

(19) United States
(12) Patent Application Publication
(10) Pub. No.: US 2009/0029917 A1
(43) Pub. Date: Jan. 29, 2009

(54) METHODS AND COMPOSITIONS FOR THE TREATMENT OF FIBROTIC CONDITIONS & IMPAIRED LUNG FUNCTION & TO ENHANCE LYMPHOCYTE PRODUCTION

(76) Inventors: Aprile L. Pilon, Gaithersburg, MD (US); Richard W. Welch, Gaithersburg, MD (US); Jeffrey Farrow, Ellicott City, MD (US); James Melby, Mount Airy, MD (US); Laura Wiese, Germantown, MD (US); Gerald Lohnas, Mount Airy, MD (US); Lucio Miele, West Chicago, IL (US); Gianni Antico, Oak Park, IL (US)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(21) Appl. No.: 12/104,295

(22) Filed: Apr. 16, 2008

Related U.S. Application Data
(63) Continuation of application No. 11/189,229, filed on Jul. 25, 2005, now abandoned.
Application No. 11/189,229 is a continuation of application No. 09/835,784, filed on Apr. 13, 2001, now abandoned.
Application No. 09/835,784 is a continuation-in-part of application No. 09/549,926, filed on Apr. 14, 2000, now abandoned.
Application No. 09/549,926 is a continuation-in-part of application No. 09/120,264, filed on Jul. 21, 1998, now abandoned.
Application No. 09/120,264 is a continuation-in-part of application No. 09/087,210, filed on May 28, 1998, now abandoned.
Application No. 09/087,210 is a continuation-in-part of application No. 08/864,357, filed on May 28, 1997, now Pat. No. 6,255,281.

(51) Int. Cl. A61K 38/17

(52) U.S. Cl. 514/12

(57) ABSTRACT

The present invention provides methods and compositions to treat fibrotic conditions, to increase lymphocyte production in vivo, and to improve and/or normalize lung function, pulmonary compliance, blood oxygenation, and blood pH to inhibit inflammatory processes to stimulate or inhibit pro-inflammatory and immune cells, and to inhibit migration of vascular endothelial cells. The invention contemplates the administration of human uteroglobin, native or recombinant, as a means of achieving these ends. Specifically, it has been found that uteroglobin inhibits cell adhesion to fibronectin, increases lymphocyte production in vivo, and improves and/or normalizes lung function, pulmonary compliance, blood oxygenation, and blood pH, and inhibits inflammatory process. In addition it has been found that uteroglobin can stimulate or inhibit pro-inflammatory and immune cells and inhibitor migration of vascular endothelial cells.

### CROSS-REFERENCE TO RELATED APPLICATIONS

This application is a continuation of U.S. application Ser. No. 11/189,229, filed Jul. 25, 2005 which is a continuation of U.S. application Ser. No. 09/835,784, filed Apr. 13, 2001, which is a continuation in part of the following applications: U.S. application Ser. No. 09/549,926, filed Apr. 14, 2000, which is a continuation-in-part of U.S. application Ser. No. 09/120,264, filed Jul. 21, 1998, which is a continuation-in-part of U.S. application Ser. No. 09/087,210, filed May 28, 1998, which is a continuation-in-part of U.S. application Ser. No. 08/864,357, filed May 28, 1997. The disclosures of each of the aforementioned applications are incorporated herein by reference.

### FIELD OF THE INVENTION

The present invention relates to the use of human uteroglobin or recombinant human uteroglobin in the treatment of fibrotic conditions, to increase lymphocyte production in vivo, to improve and/or normalize lung function, pulmonary compliance, blood oxygenation, and blood pH to inhibit inflammatory processes, to stimulate or inhibit pro-inflammatory and immune cells, and to inhibit migration of vascular endothelial cells. Novel physiological roles and therapeutic targets for uteroglobin have been identified. Specifically, the invention provides a method of inhibiting cell adhesion to fibronectin by administering human uteroglobin or recombinant human uteroglobin. The invention also provides a method of increasing lymphocyte production in vivo by administering human uteroglobin or recombinant human uteroglobin. In addition, the invention provides a method of improving lung function by administering human uteroglobin or recombinant human uteroglobin. Further the invention provides a method of inhibiting inflammatory processes by administering human uteroglobin or recombinant human uteroglobin. The invention also provides a method of stimulating or inhibiting pro-inflammatory and immune cells by administering human uteroglobin. In addition the invention provides a method of inhibiting the migration of vascular endothelial cells by administering human uteroglobin or recombinant human uteroglobin. The invention also provides a bioinformatics approach to the identification of human uteroglobin receptor(s).

Documents cited in this application relate to the state-of-the-art to which this invention pertains. The disclosures of each of these references are incorporated herein by reference.

### BACKGROUND OF THE INVENTION

Uteroglobin (also known as UG, CC10, CC16, CC17, urine protein-1, P-1, progesterone binding protein, PCB-binding protein, Clara cell secretory protein (CCSP), blastokinin, retinol-binding protein, phospholipid-binding protein, and alpha2-microglobulin) is a highly conserved mammalian protein that is primarily produced by the pulmonary epithelia. It is present in the mucosal fluid of the respiratory tract, circulates in the blood, and is excreted in the urine. Uteroglobin is a small globular homodimeric protein that consists of two identical seventy amino acid peptides that complex in an anti-parallel orientation. It has a molecular weight of 15.8 kDa, but it migrates in electrophoretic gels at a size corresponding to 10 kDa. Two disulfide bonds spontaneously form to covalently link the monomers as a dimer. Human uteroglobin is abundant in the adult human lung, and comprises up to about 7% of the total soluble protein. However, its expression is not fully activated in the developing human fetus until late in gestation. Consequently, the extracellular lung fluids of pre-term infants contain far less human uteroglobin than those of adults. Uteroglobin is also expressed by the pancreas.

Amino acid analysis of purified human uteroglobin reveals that it is structurally similar but not identical to other uteroglobin-like proteins, e.g. rabbit uteroglobin; 39 of 70 amino acids are identical between human and rabbit uteroglobin. The uteroglobin-like proteins, including human uteroglobin, rat uteroglobin, mouse uteroglobin, and rabbit uteroglobin, exhibit species-specific and tissue-specific antigenic differences, as well as differences in their tissue distribution and biochemical activities in vitro. Uteroglobin-like proteins have been described in many different contexts with regard to tissue and species of origin, including rat lung, human urine, sputum, blood components, rabbit uterus, rat and human prostate, and human lung.

The absence of structural identity among uteroglobin-like proteins makes it impossible to predict whether a protein will possess in vivo therapeutic function in humans based on in vitro or other activity exhibited by a structurally related protein. For example, human uteroglobin binds less than 5% of the amount of progesterone as rabbit uteroglobin binds in the same assay. In addition, human uteroglobin has a lower isoelectric point (4.7) than rabbit uteroglobin (5.4).

Uteroglobin is known to inhibit the enzymatic activity of secretory (soluble) phospholipases A2 (sPLA₂s) which hydrolyze phospholipids, sometimes releasing arachidonic acid in the process. Arachidonic acid is a precursor for several pro-inflammatory and anti-inflammatory eicosanoids. The role of uteroglobin as an anti-inflammatory agent in vivo was confirmed by the discovery of an inflammatory phenotype in the organs of a transgenic uteroglobin knockout mouse (U.S. Ser. No. 08/864,357). The renal fibrotic phenotype of the uteroglobin knockout mouse also led to the discovery that uteroglobin forms a complex with fibronectin, preventing fibronectin aggregation and deposition in vivo (U.S. Ser. No. 08/864,357). In addition, it was found that uteroglobin prevents the formation of a complex between fibronectin and IgA. However, this animal exhibits no pulmonary phenotype.

The renal fibrotic phenotype of the uteroglobin knockout mouse first disclosed in U.S. Ser. No. 08/864,357 led to the discovery that uteroglobin may play a significant role in controlling fibronectin aggregation and deposition. Fibronectin is a 200 kDa glycoprotein which exists in several different forms and is secreted by different tissues. Fibronectin is an essential protein and targeted disruption of the fibronectin gene in mice showed that it has a central role in embryogenesis. Fibronectin also plays a key role in inflammation, cell adhesion, tissue repair and fibrosis, and is deposited at the site of injury. Plasma fibronectin is secreted by the liver and circulates in the plasma. In the lung, cellular fibronectin is secreted upon inflammation and injury. Both types of fibronectin are chemotactic factors for inflammatory cells and fibroblasts. They also interact with cell surface proteins, called integrins, as well as cell adhesion molecules to anchor cells during adhesion and extravasation. Large numbers of inflammatory cells and fibroblasts infiltrate the lung during inflammatory episodes, which can lead to pulmonary fibrosis and ultimately death. Elevated levels of fibronectin have been detected in human clinical conditions such as neonatal respiratory distress syndrome and bronchopulmonary disease of the lung, and glomerular nephropathy of the kidney.

However, the physiological role of uteroglobin remains a source of controversy in the art. Stripp et al. (1996) also generated a uteroglobin knockout mouse in which the expression of uteroglobin was eliminated. The mouse has Clara cells which exhibit odd intracellular structures in place of uteroglobin secretion granules, but there is no other life-threatening phenotype. This knockout mouse also showed no evidence of renal, pancreatic, or reproductive abnormality. These results are completely at odds with the observations made from the uteroglobin knockout mouse described in U.S. Ser. No. 08/864,357. This mouse does, however, exhibit exacerbated pulmonary inflammation when challenged with pulmonary insult.

Leyton et al. (1994) reported the anti-metastatic properties of uteroglobin which were attributed to its inhibition of the release of arachidonic acid by tumor cells. (See also U.S. Pat. No. 5,696,092 to Patierno et al.) Kundu et al. (1996) continued this work with the observation of inhibition of extracellular matrix invasiveness by a variety of tumor cell types. Extracellular matrix invasion correlated with the presence of a 190 kDa uteroglobin binding protein in responsive cell types. The extracellular matrix invasion activity of cells lacking this protein could not be inhibited by uteroglobin.

New investigations into the therapeutic properties of uteroglobin in non-murine animal models has led to the discovery of novel mechanisms of action in vivo that are distinct from the effects of uteroglobin on inflammation and fibrosis previously observed by skilled artisans in the field.

### OBJECT OF THE INVENTION

It is an object of the present invention to provide a method of improving and/or normalizing lung function, pulmonary compliance, blood oxygenation, and/or blood pH by administering an effective amount of human uteroglobin or recombinant human uteroglobin.

It is also an object of the invention to provide a composition consisting of an amount of human uteroglobin or recombinant human uteroglobin sufficient to improve and/or normalize lung function, pulmonary compliance, blood oxygenation, and/or blood pH. Such a composition should include a pharmaceutically acceptable carrier or diluent, and the composition should preferably consist of dimeric recombinant human uteroglobin containing two disulfide bridges.

Further, it is an object of the invention to provide a method of increasing lymphocyte production in vivo by administering an amount of human uteroglobin or recombinant human uteroglobin sufficient to increase lymphocyte production and/or decrease polymorphonuclear leukocyte proliferation. Preferably, the concentration of effector lymphocytes and/or cytotoxic T cells is increased by the administration of uteroglobin. Moreover, it is an object of the invention to administer uteroglobin to increase lymphocyte production and/or decrease polymorphonuclear leukocyte proliferation in patients suffering from an autoimmune disease or allergy.

It is an additional object of the invention to provide a composition consisting of an amount of human uteroglobin or recombinant human uteroglobin sufficient to increase lymphocyte production and/or decrease polymorphonuclear leukocyte proliferation, together with a pharmaceutically acceptable carrier or diluent.

Still further, it is an object of the present invention to provide a method of inhibiting cellular adhesion to fibronectin by administering an amount of human uteroglobin or recombinant human uteroglobin sufficient to inhibit cellular adhesion to fibronectin in vivo. It is a further object of the invention to inhibit inflammatory cell and fibroblast migration on fibronectin already deposited in vivo, and to inhibit the interaction between a cell and an extracellular matrix protein and/or membrane bound protein.

Another object of the present invention to provide a composition consisting of an amount of human uteroglobin or recombinant human uteroglobin sufficient to inhibit cellular adhesion to fibronectin in vivo. Such compositions should consist of a pharmaceutically acceptable carrier or diluent.

It is an additional object of the invention to provide a method of inhibiting inflammatory processes by administering to a patient an amount of human uteroglobin or recombinant uteroglobin to inhibit inflammatory processes. Further, it is an object of the invention to provide a composition consisting of an amount of human uteroglobin or recombinant human uteroglobin inflammatory processes.

Still further, it is an object of the invention to provide a method of method of stimulating or inhibiting pro-inflammatory and immune cells by in vivo or in vitro using an amount of human uteroglobin or recombinant human uteroglobin to stimulate or inhibit such cells.

It is another object of the invention to provide a composition consisting of an amount of human uteroglobin or recombinant human uteroglobin sufficient to stimulate or inhibit pro-inflammatory and immune cells in vivo or in vitro.

Still further, it is an object of the invention to provide a method of inhibiting the migration of vascular endothelial cells by administering an amount of human uteroglobin or recombinant human uteroglobin sufficient to inhibit vascular endothelial cell migration. Another object of the invention is to provide a composition consisting of an amount of human uteroglobin or recombinant human uteroglobin sufficient to inhibit endothelial cell migration.

An additional object of the invention is to provide a bioinformatics approach to identify human uteroglobin receptor(s).

### SUMMARY OF THE INVENTION

It has now been found that uteroglobin plays a central physiological role in fibronectin deposition, lymphocyte production, smooth muscle function, and lung function in vivo.

In a first experiment it was found that the administration of uteroglobin to neonatal lambs delivered by caesarian section, an accepted model for surfactant-dependent neonatal respiratory distress syndrome in humans, led to improved and/or normalized blood oxygenation and pH. These effects are indicative of improved lung function. This observation, discussed in more detail below, is the first observation of the direct effect of uteroglobin on lung tissue and the first indication that uteroglobin may be used to improve and/or normalize lung function.

Further, in a second experiment using newborn piglets, it was found that the administration of recombinant human uteroglobin increased pulmonary compliance. The newborn piglet is an excellent model for neonatal lung injury mediated by oxygen toxicity arising from the use of positive pressure ventilation and elevated oxygen delivery in respiratory distress syndrome rescue. This significant observation was the first indication that uteroglobin may be used to increase pulmonary compliance. This effect was independent of any effects of uteroglobin on surfactant function. These data show that uteroglobin may be used to treat patients suffering from reduced pulmonary compliance as a result of a pulmonary challenge or insult resulting from exposure to non-atmospheric gases, inhaled chemicals, pollutants, irritants, pollens, allergens, particulate matter, and airborne infectious agents. It was also found that a single dose of recombinant human uteroglobin to newborn piglets significantly increased lymphocyte proliferation and decreased polymorphonuclear leukocyte proliferation. The increase in lymphocyte proliferation was significant, up to 2.5 fold, and the decrease in polymorphonuclear leukocyte proliferation of up to 2.3 fold persisted for a period exceeding one month.

In addition, using two new assay formats designed to specifically detect uteroglobin-fibronectin binding, it was found that recombinant human uteroglobin binds to portions of fibronectin that are important in cell adhesion and not known to be relevant to fibrillogenesis. Fibronectin consists of eight type I domains in the N-terminal third of the protomer, three type I domains at the C-terminus, two type II domains clustered in the middle of the protomer, and 15-17 type III domains, depending on the tissue of origin. One or more of the type III domains have been implicated in cell adhesion, fibronectin-fibronectin interactions, and deposition in vitro. Using two commercially available chymotryptic fragments of fibronectin, each containing type III domains involved in fibronectin-dependent cell adhesion and/or polymerization, and a recombinant fragment of fibronectin, termed “superfibronectin” (so named because of its ability to promote fibronectin-fibronectin interactions, polymerization, deposition, and cell adhesion in vitro), the interaction between uteroglobin and the various regions of fibronectin was examined.

A clear dose-response relationship in binding between recombinant human uteroglobin and “superfibronectin” was observed. This indicates that recombinant human uteroglobin binds to the type III domain of fibronectin which is represented by “superfibronectin”. Further, it was also found that recombinant human fibronectin binds to more than one type III domain of fibronectin because a dose-response relationship was observed for binding between uteroglobin and a chymotryptic fragment that does not contain the “superfibronectin” domain. Still further, because fibronectin type III domains are present in nearly all components of the extracellular matrix, e.g., laminin, collagens, vitronectin, and fibrin, as well as in numerous membrane bound proteins, e.g., adhesion molecules, integrins, and receptors, these domains may play a central role in cell-cell and cell-extracellular matrix interactions. The observation that uteroglobin interacts with these domains shows that it may be used to mediate these interactions and physiological conditions affected by such interactions.

Therefore, according to one aspect of the present invention, the invention provides a method of treatment including improving and/or normalizing lung function in a patient in need of such treatment, wherein the method consists of administering an amount of uteroglobin effective to improve and/or normalize lung function relative to that observed in the absence of such treatment. In a preferred embodiment, the uteroglobin is recombinant human uteroglobin, and the preferred dosage range is 10 ng/kg-25 mg/kg.

According to a further aspect, the invention provides a method of improving and/or normalizing pulmonary compliance in a patient in need of such treatment, wherein the method consists of administering an amount of uteroglobin to the patient sufficient to improve and/or normalize pulmonary compliance relative to that observed in the absence of such treatment. In a preferred embodiment, the uteroglobin is recombinant human uteroglobin, and the preferred dosage range is 10 ng/kg-25 mg/kg. The reduced pulmonary compliance may have resulted from pulmonary challenge or insult resulting from exposure to non-atmospheric gases, inhaled chemicals, pollutants, irritants, inhaled pollens, allergens, particulate matter, and airborne infectious agents.

An additional aspect of the invention provides a method of treating a patient suffering from reduced blood oxygenation and/or blood pH, wherein the method includes administering an amount of uteroglobin effective to improve and/or normalize blood oxygenation and/or blood pH relative to that observed in the absence of such treatment. In a preferred embodiment, the uteroglobin is recombinant human uteroglobin, and the preferred dosage range is 10 ng/kg-25 mg/kg.

Another aspect of the invention provides compositions consisting of uteroglobin effective for the improvement and/or normalization of lung function, pulmonary compliance, blood oxygenation, and/or blood pH. Such compositions preferably contain a dosage of 10 ng-500 mg, in combination with a pharmaceutically acceptable carrier or diluent, wherein the amount of uteroglobin contained in the composition is commensurate with the administration of 10 ng/kg-25 mg/kg by the method of the present invention. In a preferred embodiment, the uteroglobin is recombinant human uteroglobin.

According to a further aspect, the invention provides a method of increasing lymphocyte production in a patient in need of such treatment, wherein the method includes administering an amount of uteroglobin sufficient to increase lymphocyte production in the patient relative to that observed in the absence of such treatment. In a preferred embodiment, recombinant human uteroglobin is used at a dosage range of 1 ng/kg-100 mg/kg. Preferably, the method increases the production of effector lymphocytes and/or cytotoxic T cells. The patient may be suffering from decreased lymphocyte production as a result of an autoimmune disease, such as acquired immunodeficiency syndrome, or an allergy. In addition, uteroglobin may be used to enhance a lymphocyte response to a vaccine.

In an additional aspect, the invention provides a method of increasing the production of suppressor T cells in a patient in need of such treatment, wherein the method includes administering an amount of uteroglobin sufficient to increase production of suppressor T cells in the patient relative to that observed in the absence of such treatment. In a preferred embodiment, recombinant human uteroglobin is used at a dosage range of 1 ng/kg-100 mg/kg.

In accordance with an additional aspect, the invention provides a method of enhancing a lymphocyte-mediated response in a patient in need of such treatment, wherein the method includes administering an amount of uteroglobin sufficient to enhance such a response in the patient relative to that observed in the absence of such treatment. In a preferred embodiment, recombinant human uteroglobin is used at a dosage range of 1 ng/kg-100 mg/kg.

A further aspect of the invention is to provide a method of decreasing the production of polymorphonuclear leukocytes in a patient in need of such treatment, wherein the method includes administering an amount of uteroglobin sufficient to decrease polymorphonuclear leukocyte production in the patient relative to that observed in the absence of such treatment. In a preferred embodiment, recombinant human uteroglobin is used at a dosage range of 1 ng/kg-100 mg/kg.

An aspect of the invention is to provide compositions including uteroglobin in an amount sufficient to increase production of lymphocytes and/or suppressor T cells, enhance a lymphocyte-mediated response, and/or to decrease the production of polymorphonuclear leukocytes in a patient in need of such treatment. In a preferred embodiment, recombinant human uteroglobin is used in the composition at a dosage range of 1 ng/kg-100 mg/kg, together with a pharmaceutically acceptable carrier or diluent.

According to an additional aspect, the invention provides a method of treatment including inhibiting fibronectin-dependent cell adhesion to fibronectin in a patient in need of such treatment, wherein the method consists of administering an amount of uteroglobin to the patient sufficient to inhibit fibronectin-dependent cell adhesion to fibronectin relative to that observed in the absence of such treatment. In a preferred embodiment, the uteroglobin is recombinant human uteroglobin, and the preferred dosage range is 8 μg-3.5 g dose per 70 kg patient. Most preferably, the dosage range is 25 ng/200 μl-10 μg/200 μl. In an additional preferred embodiment, uteroglobin blocks cell adhesion to type III domains of fibronectin.

An aspect of the invention provides a method of treatment including inhibiting an interaction between fibronectin and cells dependent on fibronectin binding, wherein the method includes administering an amount of uteroglobin effective to inhibit such interactions relative to that observed in the absence of such treatment. In a preferred embodiment, the uteroglobin is recombinant human uteroglobin, and the preferred dosage range is 8 μg-3.5 g dose per 70 kg patient. Most preferably, the dosage range is 25 ng/200 μl-10 μg/200 μl.

According to a further aspect, the invention provides a method of inhibiting inflammatory cell and fibroblast migration on fibronectin already deposited in vivo in a patient in need of such treatment, wherein the method includes administering an amount of uteroglobin sufficient to inhibit fibronectin-dependent cell adhesion to fibronectin in the patient relative to that observed in the absence of such treatment. In a preferred embodiment, the uteroglobin is recombinant human uteroglobin, and the preferred dosage range is 8 μg-3.5 g dose per 70 kg patient. Most preferably, the dosage range is 25 ng/200 μl-10 μg/200 μl.

In another aspect, the invention provides a method of inhibiting fibronectin-dependent cell adhesion in a patient in need of such treatment, wherein the method includes administering an amount of uteroglobin sufficient to inhibit fibronectin-dependent cell adhesion in the patient relative to that observed in the absence of such treatment.

In accordance with an aspect, the invention provides a method of inhibiting an interaction between a cell and an extracellular matrix protein and/or membrane bound protein in a patient in need of such treatment, wherein the method includes administering an amount of uteroglobin sufficient to inhibit such interactions in the patient relative to that observed in the absence of such treatment. In a preferred embodiment, the uteroglobin is recombinant human uteroglobin, and the preferred dosage range is 8 μg-3.5 g dose per 70 kg patient. Most preferably, the dosage range is 25 ng/200 μl-10 μg/200 μl.

Another aspect of the invention is to provide compositions including uteroglobin in an amount sufficient to inhibit fibronectin-dependent cell adhesion to fibronectin, an interaction between fibronectin and cells dependent on fibronectin binding, inflammatory cell and fibroblast migration on fibronectin deposited in vivo, fibronectin-dependent cell adhesion, and an interaction between a cell and an extracellular matrix protein and/or membrane bound protein in a patient in need of such treatment. In a preferred embodiment, recombinant human uteroglobin is used in the composition, together with a pharmaceutically acceptable carrier or diluent, and the preferred dosage range is 8 μg-3.5 g dose per 70 kg patient. Most preferably, the dosage range is 25 ng/200 μl-10 μg/200 μl.

Another aspect of the invention is to provide methods and compositions to inhibit an LPS-dependent inflammatory processes in a patient infected with a bacterium by administering to a patient an amount of recombinant human uteroglobin sufficient to inhibit the inflammatory processes.

An additional aspect of the invention is to provide methods and compositions to decrease TNF-alpha concentrations in vivo in a patient in need of such treatment by administering to a patient an amount of recombinant human uteroglobin sufficient to decrease TNF-alpha concentrations.

A further aspect of the invention is to provide methods and compositions to regulate the nitric oxide pathway for relaxing smooth muscle cells in a patient in need of such treatment by administering to the patient an amount of recombinant human uteroglobin sufficient to regulate the nitric oxide pathway.

Another aspect of the invention is to provide methods and compositions to regulate vascular permeability in a patient in need of such treatment by administering to the patient an amount of recombinant human uteroglobin sufficient to regulate vascular permeability.

A further aspect of the invention is to provide methods and compositions to of suppress proliferation of CD71-positive cells in a patient in need of such treatment by administering to the patient an amount of recombinant human uteroglobin sufficient to suppress proliferation of such cells.

An additional aspect of the invention is to provide methods and compositions to suppress proliferation of CD71-positive cells in vitro by exposing CD71-positive cells to an amount of recombinant human uteroglobin sufficient to suppress proliferation of the cells in vitro.

Another aspect of the invention is to provide methods and compositions to of suppress proliferation of CD71-positive cells in vitro by exposing CD71-positive cells to an amount of recombinant human uteroglobin and an amount of fibronectin sufficient to suppress proliferation of the cells in vitro.

A further aspect of the invention is to provide methods and compositions to suppress activation of CD71-positive cells in a patient in need of such treatment by administering to the patient an amount of recombinant human uteroglobin sufficient to suppress activation of such cells.

An additional aspect of the invention is to provide methods and compositions to suppress activation of CD71-positive cells in vitro by exposing the cells to an amount of recombinant human uteroglobin sufficient to suppress activation of the cells in vitro.

An additional aspect of the invention is to provide methods and compositions to enhance proliferation of CD11b-positive cells in a patient in need of such treatment by administering to the patient an amount of recombinant human uteroglobin sufficient to enhance proliferation of such cells.

Another aspect of the invention is to provide methods and compositions to enhance proliferation of CD11b-positive cells in vitro by exposing the cells to an amount of recombinant human uteroglobin sufficient to enhance proliferation of the cells in vitro.

A further aspect of the invention is to provide methods and compositions to enhance activation of CD 11b-positive cells in a patient in need of such treatment by administering to the patient an amount of recombinant human uteroglobin sufficient to enhance activation of the cells.

An additional aspect of the invention is to provide methods and compositions to enhance activation of CD11b-positive cells in vitro by exposing said cells to an amount of recombinant human uteroglobin sufficient to enhance activation of the cells in vitro.

Another aspect of the invention is to provide methods and compositions to inhibit migration of vascular endothelial cells by administering recombinant human uteroglobin to a patient in need of such treatment in an amount sufficient to inhibit migration of such cells.

A further aspect of the invention is to provide methods and compositions to inhibit angiogenesis in a patient in need of such treatment by administering to the patient an amount of recombinant human uteroglobin sufficient to inhibit angiogenesis.

An additional aspect of the invention is to provide methods and compositions to inhibit migration of vascular endothelial cells in a patient in need of such treatment by administering to the patient recombinant human uteroglobin and fibronectin or a fragment derived from fibronectin in amounts sufficient to inhibit migration of said cells.

Another aspect of the invention is to provide methods and compositions to inhibit angiogenesis in a patient in need of such treatment by administering to the patient recombinant human uteroglobin and fibronectin, or a fragment derived from fibronectin, in amounts sufficient to inhibit angiogenesis.

A further aspect of the invention is to provide methods and compositions to inhibit extracellular matrix invasion by vascular endothelial cells in a patient in need of such treatment by administering to the patient an amount of recombinant human uteroglobin sufficient to inhibit extracellular matrix invasion of such cells.

An additional aspect of the invention is to provide methods and compositions to inhibit extracellular matrix invasion by vascular endothelial cells in a patient in need of such treatment by administering to the patient recombinant human uteroglobin and fibronectin or a fragment derived from fibronectin in amounts sufficient to inhibit extracellular matrix invasion.

Another aspect of the invention is to provide methods and compositions to regulate signal transduction mediated by CD148 and CD148 immunoreactive proteins in uteroglobin-responsive cells by exposing the cells to recombinant human uteroglobin.

A further aspect of the invention is to provide methods and compositions to regulate cellular activities mediated by CD148 and CD148 immunoreactive proteins by exposing the cells to recombinant human uteroglobin.

An additional aspect of the invention is to provide methods and compositions to regulate signal transduction mediated by PLA2 receptors and PLA2 immunoreactive proteins in uteroglobin-responsive cells by exposing the cells to recombinant human uteroglobin.

Another aspect of the invention is to provide methods and compositions to regulate cellular activities mediated by CD148 and CD148 immunoreactive proteins by exposing the cells to recombinant human uteroglobin.

An additional aspect of the invention is to provide methods to identify proteins that interact with each other, in which at least one protein contains at least one four helical bundle motif and at least one protein having at least one fibronectin Type III domain by mapping a pathway involving one or more protein interactions.

### BRIEF DESCRIPTION OF THE DRAWINGS

The invention will now be described in more detail, with reference to the accompanying drawings, in which:

FIG. 1 shows the standard curve obtained using the uteroglobin (UG) immunoassay described below.

FIG. 2 shows the bicarbonate excess (BE) exhibited in pre-term lambs upon intratracheal administration of recombinant human uteroglobin.

FIG. 3 shows the decrease in CO₂ exhibited in pre-term lambs upon intratracheal administration of recombinant human uteroglobin.

FIG. 4 shows the increase in blood pH exhibited in pre-term lambs upon intratracheal administration of recombinant human uteroglobin.

FIG. 5 shows the increase in paO₂/FiO₂ exhibited in pre-term lambs upon intratracheal administration of recombinant human uteroglobin.

FIG. 6A shows the concentration of recombinant human uteroglobin (CC 10) in serum as a function of time after intratracheal administration of recombinant human uteroglobin to newborn piglets.

FIG. 6B shows the total protein concentration in BAL fluids obtained from piglets in each of the eight treatment groups.

FIG. 7 shows the pressure-volume relationship observed upon administration of recombinant human uteroglobin to newborn piglets.

FIG. 8 shows the mean pressure-volume relationship observed upon administration of recombinant human uteroglobin to newborn piglets ventilated with 100% oxygen.

FIG. 9 shows the mean pressure-volume relationships observed for all animals upon administration of recombinant human uteroglobin to newborn piglets ventilated with room air and 100% oxygen.

FIG. 10A shows the mean pressure-volume curve among four treatment groups of newborn piglets administered recombinant human uteroglobin.

FIG. 10B shows the mean pressure-volume curve among five treatment groups of newborn piglets administered recombinant human uteroglobin.

FIG. 10C shows changes in PMN and lymphocyte cell counts over a 28-day period.

FIG. 11 shows radioactive counts as a function of time for each group of Wistar rats administered recombinant human uteroglobin via intravenous administration.

FIG. 12 shows radioactive counts as a function of time for each group of Wistar rats administered recombinant human uteroglobin via intranasal administration.

FIG. 13 shows radioactive counts as a function of time for each group of Wistar rats administered recombinant human uteroglobin via stomach gavage.

FIG. 14 shows the concentration of recombinant human uteroglobin as a function of time for each group of Wistar rats administered recombinant human uteroglobin via intravenous administration.

FIG. 15 shows the concentration of recombinant human uteroglobin as a function of time for each group of Wistar rats administered recombinant human uteroglobin via intranasal administration.

FIG. 16 shows the concentration of recombinant human uteroglobin as a function of time for each group of Wistar rats administered recombinant human uteroglobin via stomach gavage.

FIGS. 17A-17B are schematic representations of two ELISA-based assay formats for the uteroglobin-fibronectin binding interaction. Format A, shown in FIG. 17A represents an assay based on immunodetection, wherein CC10 is uteroglobin and HRP is horse radish peroxidase. Format B, shown in FIG. 17B represents a competitive binding assay format in which CC10 is uteroglobin, HRP is horse radish peroxidase, and rhFn is recombinant human fibronectin, and the free uteroglobin in the same competes with the HRP-labeled uteroglobin for binding sites on recombinant human fibronectin.

FIG. 18 shows a map of the human fibronectin protomer.

FIG. 19 shows the results obtained from binding assays between uteroglobin (CC 10) and intact and fragmented fibronectin using format A, wherein hFn is human fibronectin and SuperFn is superfibronectin.

FIGS. 20A-20B show the dose response curves for uteroglobin binding to fibronectin and its fragments using format A, wherein rhUG is recombinant human uteroglobin, hFn is human fibronectin, and Fn is fibronectin.

FIG. 21 shows the mean change in airway resistance in perfused rat lung.

FIG. 22 shows the mean concentration of TNF-alpha in BAL fluid from perfused rat lung.

FIG. 23 shows a flow diagram of the maturation of hemopoietic stem cells.

FIG. 24 shows ATPase activity in CD71-positive cells from rats treated with recombinant human uteroglobin.

FIG. 25 shows ATPase activity in CD11b-positive cells from rats treated with recombinant human uteroglobin.

FIG. 26 shows an in vitro wound healing assay with recombinant human uteroglobin.

FIGS. 27A and 27B shows the inhibition of endothelial cell migration by recombinant human uteroglobin.

FIG. 28 shows an extracellular matrix invasion assay with endothelial cells and recombinant human uteroglobin.

FIG. 29A shows the endothelial cell growth curve with and without recombinant human uteroglobin.

FIG. 29B shows the dose dependent response of endothelial cell VEGF stimulated proliferation assay.

FIG. 30 shows a Western blot of proteins from three cell lines probed with anti-fibronectin antibody.

FIG. 31 shows colony counts of A549 soft agar assay.

FIG. 32 shows a silver-stained SDS-Page gel indicating UG affinity purified bands that cross react with specific antibodies.

It is noted that the patent or application file contains at least one drawing executed in color. Copies of this patent or patent application publication with the color drawing will be provided by the U.S. Patent Office upon request and payment of the necessary fee.

### DETAILED DESCRIPTION OF PREFERRED EMBODIMENTS

### Definitions

Native and recombinant human uteroglobin may be used in the present invention. In a preferred embodiment, however, recombinant human uteroglobin is employed in the methods and compositions of the invention. The recombinant form of uteroglobin preferably has substantially the same amino acid sequence as that of the native human uteroglobin protein. An amino acid sequence having “substantially the same” amino acid sequence as that of the native human protein includes recombinant human uteroglobin having at least 75% identity to the native human protein. In a preferred embodiment, recombinant human uteroglobin has at least 85% identity, and in a most preferred embodiment, recombinant human uteroglobin has at least 98% identity to the native uteroglobin, native human uteroglobin having the amino acid sequence of EICPSFQRVIETLLMDTPSSYEAAMELFSPDQDMREAGAQLKKLVDTLPQKPRESIIK LMEKIAQSSLCN (SEQ ID NO.1). In a further preferred embodiment, dimeric recombinant human uteroglobin is used in the methods and compositions of the present invention (with respect to the various forms of uteroglobin, reference is made to U.S. Ser. No. 09/120,264).

Also included in the method of the present invention is the use of fragments or derivatives of uteroglobin, native or recombinant. A “fragment” of uteroglobin refers to a portion of the native uteroglobin amino acid sequence having six or more contiguous amino acids of the native protein sequence. The term “derivative” refers to peptide analogs of uteroglobin, including one or more amino acid substitutions and/or the addition of one or more chemical moieties, e.g., acylating agents, sulfonating agents, carboxymethylation of the disulphide bonds, or complexed or chelated metal or salt ions, e.g. Mg⁺², Ca⁺², or Na⁺¹, with the proviso that the derivative retains the biological activity of the parent molecule. In addition, the present invention also contemplates the use of small molecule mimetics and chemical structural derivatives of uteroglobin.

A “uteroglobin-like” protein includes those isolated from mouse, rat, rabbit, etc., having substantially the same amino acid sequences and/or substantial sequence similarity, termed conservative substitutions, with native human uteroglobin. With regard to sequence similarity, like-amino acids may be substituted in a uteroglobin-like protein, e.g. tyrosine for phenylalanine or glycine for alanine. Uteroglobin-like proteins which are considered substantially similar have approximately 30% sequence similarity, preferably 50% sequence similarity, more preferably at least 75% sequence similarity, and most preferably at least 90-95% sequence similarity. Uteroglobin-receptor ligands are peptide, protein or chemical moieties (e.g. organic ligands) that bind to the uteroglobin receptor and mediate all or part of its activities. Uteroglobin structural analogs are compounds, peptides or proteins, or fragments or derivatives thereof having substantially similar secondary and tertiary structural characteristics when compared to native uteroglobin, such that a structural analog retains at least 50% and preferably at least 75% of the activity of native protein. In a most preferred embodiment, a structural analog retains at least 90% of the activity of the native protein and retains the ability to interact with the uteroglobin receptor and to mediate all or part of its activities. As used herein, the term “recombinant human uteroglobin” includes recombinant CC10.

Further, the uteroglobin used in the method of the present invention is substantially pure. The term “substantially pure” refers to uteroglobin having a purity of about 75% to about 100%. In a preferred embodiment, uteroglobin has a purity of about 90% to about 100%, and in the most preferred embodiment, uteroglobin has a purity of at least 95%.

In addition, as used herein “fibronectin immunoreactive protein” includes proteins, protein fragments, glycosylated or otherwise modified, peptides or derivatives that react with anti-human fibronectin monoclonal antibody.

Further, “CD148 immunoreactive protein” includes protein, protein fragments, peptides, or derivatives that react with anti-CD148 polyclonal antibody.

In addition, as used herein “PLA₂ receptor immunoreactive protein” includes protein, protein fragments, peptides, or derivatives that react with an anti M type PLA₂ receptor antibody.

In so much as the present invention provides a method of treating or preventing a disease condition associated with fibronectin deposition, lung damage, and/or decreased lymphocyte production, the term “prevention” refers to preventing the development of disease in a susceptible or potentially susceptible population, or limiting its severity or progression, whereas the term “treatment” refers to the amelioation of a disease or pathological condition. As used herein “regulate” includes increasing or decreasing in a pharmacologically, therapeutically or physiologically beneficial manner or purpose, and to enhance or re-assert therapeutically beneficial cellular and tissue control pathways.

To map a pathway means to identify the molecular participants in the pathway and to eventually determine the sequence of molecular interactions that result in a quantifiable net effect at the molecular, cellular or tissue level.

UG-like proteins are members of the UG protein family, and includes mammoglobin, lymphoglobin, lipophilins, and others, all bearing the characteristic dimeric structure with at least one monomer containing a four helical bundle motif.

Proteins that contain four helical bundle motifs include the UG and UG family, the sPLA2 protein family, the annexin family, and others.

Proteins that contain fibronectin Type III repeats include collagens, titins, tenascins, cytotactins, fibrin, cell adhesion molecules, integrins, protein tyrosine phosphatases, and others.

### The Effect of Uteroglobin in Lung Damage, Lymphocyte Production, and Fibronectin

Using neonatal lambs delivered by cesarean section as a model of surfactant-dependent neonatal respiratory distress syndrome (RDS), it was found that recombinant human uteroglobin did not interfere with surfactant replacement therapy. In fact, an animal with severe meconium aspiration which potently inactivates surfactant responded to the administration of recombinant human uteroglobin by a marked increase in blood oxygenation and pH, both of which are indicators of improved lung function. This was the first observation of a direct effect of uteroglobin on lung tissue.

It was also unexpectedly found that the bioavailability of recombinant human uteroglobin in this experiment was excellent. These results show that recombinant human uteroglobin may be administered systemically via the lungs, for the purpose of raising circulating levels of the protein, to deliver the protein to tissues and organs, and to raise the concentration of uteroglobin in the urine. Therefore, it is possible to treat various internal organs and tissues, including the vasculature, muscle, connective tissue, bone, blood cells, stomach, kidneys, pancreas, liver, intestines, colon, heart, spleen, thymus, uterus, and bladder, by administering uteroglobin topically to the lungs through intratracheal deposition or through an inhaler or nebulizer. Further, results obtained from the intravenous, intranasal, and stomach gavage administration of uteroglobin to adult Wistar rats indicated that these routes may be practical for the systemic administration of the protein in humans. The presence of radioactive recombinant human uteroglobin in protein extracts of the trachea, bronchi, esophagus, and thyroid in an animal administered uteroglobin via each of these routes show that these tissues take up uteroglobin from the circulatory system. This demonstrates that one route of administration may be effective in the specific delivery of protein to target delivery for another system, e.g., the digestive system.

Next, using a ventilated newborn piglet, a well-characterized model of neonatal lung injury, one can readily observe significant decreases in pulmonary compliance and lung function, as well as increases in inflammatory markers which are indicative of pulmonary inflammation. These animals were administered uteroglobin, then broncho-alveolar lavage fluid (BAL), serum, and urine were collected in order to monitor the half-life and elimination of uteroglobin following intratracheal administration. Further, the total protein concentration in BAL samples was measured and total soluble protein was calculated. These parameters are significant indicators of lung injury. The data show that administration of recombinant human uteroglobin increased pulmonary compliance in the newborn piglets sampled, and that the effect was independent of surfactant function. Complete blood counts (CBC) and differential cell counts were taken on the blood samples collected prior to and following uteroglobin administration to the ventilated newborn piglets. It was found that a single dose of recombinant human uteroglobin in the newborn piglets significantly enhanced lymphocyte proliferation and decreased polymorphonuclear leukocyte production.

Therefore, the present invention provides methods and compositions for improving and/or normalizing lung function, pulmonary compliance, blood oxygenation and/or blood pH. Suitable compositions include uteroglobin, and preferably recombinant human uteroglobin, in a dosage of 10 ng/kg-25 mg/kg. The methods may be used to treat patients suffering from reduced lung function and/or pulmonary compliance as a result of exposure to non-atmospheric gases, non-atmospheric ratios of atmospheric gases, inhaled chemicals, pollutants, irritants, inhaled pollens, allergens, particulate matter, and airborne infectious agents.

The present invention also provides methods and compositions for increasing lymphocyte production in vivo, increasing the production of suppressor T cells, enhancing a lymphocyte-mediated response in vivo, and decreasing the production of polymorphonuclear leukocytes. The method of the present invention contemplates a dosage of uteroglobin, preferably recombinant human uteroglobin, of 1 ng/kg-100 mg/kg. The lymphocytes that are typically affected by this method are effector lymphocytes and cytotoxic T cells, and more particularly, helper T cells, suppressor T cells, NK cells, plasma B cells, memory B cells, and their precursors. Further, in so much as the present invention provides a method of enhancing a lymphocyte-mediated response in vivo, such a method may be used to enhance the effects of the administration of a vaccine, such as a B cell or T cell vaccine, or a tolerance-inducing treatment, such as oral tolerance or allergy shots.

Finally, the interaction between uteroglobin and fibronectin was examined. It was found that recombinant human uteroglobin was a potent inhibitor of cellular adhesion to fibronectin and that it specifically bound to type III domains of fibronectin. Such domains are present in nearly all protein components of the extracellular matrix, e.g., laminin, collagens, vitronectin, and fibrin, as well as in numerous membrane bound proteins, including adhesion molecules, integrins, and receptors. Thus, the inhibition of cellular adhesion by uteroglobin indicates that uteroglobin can play a critical role in cell-cell and cell-extracellular matrix interactions.

Therefore, the present invention provides methods and compositions for inhibiting the following processes: (1) fibronectin-dependent cell adhesion to fibronectin, (2) interactions between fibronectin and cells dependent on fibronectin binding, (3) inflammatory cell and fibroblast migration on fibronectin already deposited in vivo, (4) fibronectin-dependent cell adhesion in vivo, (5) an interaction between a protein cell containing a four helical bundle motif and a protein/ECm protein containing at least one FnIII domain and (6) an interaction between a cell having a PLA₂ receptor and an extracellular matrix protein and/or membrane bound protein comprising at least one fibronectin type III domain. The compositions contain uteroglobin, and preferably recombinant human uteroglobin, in a dosage of 8 μg-3.5 g total dose per 70 kg patient, and more preferably, 25 ng/200 μl-10 μg/200 μl.

Cells dependent on fibronectin binding include, but are not limited to, neural cells, muscle cells, hematopoietic cells, fibroblasts, neutrophils, eosinophils, basophils, macrophages, monocytes, lymphocytes, platelets, red blood cells, endothelial cells, stromal cells, dendritic cells, mast cells, and epithelial cells. A patient suffering from one of the following conditions may benefit from therapies which inhibit the formation of vascular adhesions following surgery, atheroschlerosis, thrombosis, heart disease, vasculitis, formation of scar tissue, restenosis, phlobitis, COPD (chronic obstructive pulmonary disease), pulmonary hypertension, pulmonary fibrosis, pulmonary inflammation, bowel adhesions, bladder fibrosis and cystitis, fibrosis of the nasal passages, sinusitis, inflammation mediated by neutrophils, and fibrosis mediated by fibroblasts.

In so much as the invention provides a method of inhibiting inflammatory cell and fibroblast migration on fibronectin already deposited in vivo, inflammatory cells targeted by this method include, but are not limited to, neutrophils, eosinophils, basophils, macrophages, monocytes, lymphocytes, platelets, red blood cells, dendritic cells, mast cells, stem cells and fibroblasts.

Finally, extracellular matrix proteins affected by the method of the present invention include, but are not limited to, laminin, collagen, vitronectin, and fibrin, and membrane bound proteins affected by the instant method include, but are not limited to, adhesion molecules, integrins, and receptors.

Without wishing to be bound by any particular theory, the interactions between uteroglobin and lung damage, lymphocyte production, and fibronectin do not appear to be discrete and insular occurrences. Rather, they are indicative of a complex network of interactions between a variety of physiological events. First, the primary site of action of surfactant phospholipids in the lungs is thought to be the alveoli, which are membranous sacs at the end of small passages called bronchioles. The surfactant allows the alveoli to expand and fill with air in response to the expansion of the chest wall which is mediated by smooth muscle. Surfactant also mediates O₂—CO₂ gas exchange across the mucosal fluid layer and alveolar membranes. The smooth muscle components of the lungs, i.e., the endothelial cell layers of bronchi, bronchioles, blood vessels, and capillaries are not known to be affected by surfactant.

The effect of uteroglobin on pulmonary compliance and serum protein leakage into BAL fluids in piglets indicate that a second mechanism of action for uteroglobin is at work, and one that is distinct from the inhibition of PLA₂-mediated digestion of surfactant phospholipids. First, the observation that piglets ventilated with 100% oxygen that received uteroglobin had normalized lung compliance in comparison to those similarly ventilated but not administered uteroglobin indicates that uteroglobin mediates an entirely unanticipated effect on the lungs that is distinct from its protective effects on exogenous surfactant. Further, there was no significant difference in surfactant function in BAL of animals that did and did not receive uteroglobin. This shows that the uteroglobin-mediated protection of surfactant phospholipids from digestion by soluble, secreted PLA₂s did not persist 48 hours after administration in this model. In contrast, the uteroglobin-mediated effect on pulmonary compliance did persist 48 hours after administration. Thus, at the 48-hour endpoint, surfactant function did not correlate with the uteroglobin-mediated differences in pulmonary compliance. Therefore, uteroglobin mediates a surfactant-independent effect on pulmonary compliance. In accordance with these observations, uteroglobin could be described as a bronchodilator.

Because surfactant improves pulmonary compliance primarily by making the alveolar sacks more elastic, it follows that uteroglobin affects the flexibility of the other primary structures involved in pulmonary compliance, i.e., the bronchi and bronchioles. Bronchi and bronchioles are composed of three main cellular layers: the surface epithelia, the stroma, and the endothelia. The endothelial layers contain the smooth muscle responsible for the changes in volumetric capacity of the bronchi and bronchioles. Therefore, uteroglobin most likely affects pulmonary compliance by increasing the ability of smooth muscle to expand and contract. This explanation is supported by the observation that the protein content of the BAL of uteroglobin-treated piglets is significantly lower than that of untreated piglets. The source of excess protein in BAL fluids is generally the serum. The amount of protein that leaks from the serum into the BAL depends upon local vascular permeability. Pro-inflammatory treatments to the lungs, such as 100% oxygen exposure, generally increase vascular permeability, resulting in excess protein in BAL fluids. The administration of uteroglobin countered this effect. Vascular permeability is, in part, dependent upon the degree of smooth muscle contraction in blood vessels and smooth muscle contraction is controlled by the autonomic nerve system.

Fibronectin type III repeats are found in structural proteins of the musculature, such as collagen, titins, and tenascins, as well as in several cellular adhesion molecules, such as ICAM-1 (intercellular cell adhesion molecule), LFA (leukocyte function associated antigens), VCAM-1 (vascular cell adhesion molecule), and NCAMs (neural cell adhesion molecules).

There are 17 type III repeats in fibronectin that can form intramolecular and intermolecular bridges and their formation may be calcium dependent. Thus, the type III repeats can act like building blocks that fit together. When fibronectin mediates fibrillogenesis, these repeats may interact via hydrophobic and ionic interactions, acting like beads on a strand that can interlock and align. The strings may be locked into threads by the action of transglutaminase which can covalently crosslink the fibronectin strands together and build the fibril. Therefore, one can assume that fibronectin type III repeats in molecules other than fibronectin may also interact non-covalently to affect communication. For example, the interaction between fibronectin type III repeats in neural cell adhesion molecules and fibronectin type III repeats in muscle structural proteins like collagen, titins, and tenascins, could help to trigger the signal for muscle contraction. Alignment of the fibronectin type III repeats, therefore, provides a physical connection between nerve and muscle, and interactions between fibronectin type III repeats may be important to the normal function of the lungs. The enhanced pulmonary compliance in uteroglobin-treated piglets suggests that uteroglobin binds fibronectin type III repeats and may block the alignment of type III repeats in both nerve and muscle in vivo, perhaps regulating the degree of muscle contraction by sterically blocking the alignment of excess fibronectin type III repeats, in muscle tissue and at the interface of muscle tissue and neural tissue.

### Preferred Routes of Administration and Formulations for Uteroglobin

Uteroglobin may be administered either alone or in combination with other active agents or compositions typically used in the treatment or prevention of the above-identified disease conditions. Such active agents or compositions include, but are not limited to steroids, non-steroidal anti-inflammatories drugs (NSAIDs), chemotherapeutics, analgesics, immunotherapeutics, antiviral agents, antifungal agents, vaccines, immunosuppressants, hematopoietic growth factors, hormones, cytokines, antibodies, antithrombotics, cardiovascular drugs, or fertility drugs. Also included are vaccines, oral tolerance drugs, vitamins and minerals.

Uteroglobin may be administered to target a uteroglobin-receptor. Targeting of a uteroglobin receptor refers to inducing specific binding of a ligand to a receptor to mediate effects on cell growth and/or activity.

As discussed above, the data show that recombinant human uteroglobin may be administered systemically via the lungs, for the purpose of raising circulating levels of the protein, to deliver the protein to tissues and organs, and to raise the concentration of uteroglobin in the urine. Therefore, one can treat various internal organs and tissues, including the vasculature, muscle, connective tissue, bone, blood cells, stomach, kidneys, pancreas, liver, intestines, colon, heart, spleen, thymus, uterus, and bladder, by administering uteroglobin topically to the lungs through intratracheal deposition or through an inhaler or nebulizer. Further, the data also show that intravenous, intranasal, and stomach gavage administration of uteroglobin are practical for the systemic administration of the protein in humans.

Uteroglobin may be administered intravenously or, in the case of treatment of neonatal RDS/BPD and adult RDS, in the form of a liquid or semi-aerosol via the intratracheal tube. Other viable routes of administration include topical, ocular, dermal, transdermal, anal, systemic, intramuscular, subcutaneous, slow release, oral, vaginal, intraduodenal, intraperitoneal, and intracolonic. Such compositions can be administered to a subject or patient in need of such administration in dosages and by techniques well known to those skilled in the medical, nutritional or veterinary arts taking into consideration such factors as the age, sex, weight, and condition of the particular subject or patient, and the route of administration. The compositions of the present invention may also be administered in a controlled-release formulation. The compositions can be co-administered or sequentially administered with other active agents, again, taking into consideration such factors as the age, sex, weight, and condition of the particular subject or patient, and, the route of administration.

Further, the data show that recombinant human uteroglobin may be administered systemically via the digestive tract (orally), for the purpose of raising circulating levels of the protein, to deliver the protein to tissues and organs, and to raise the concentration of uteroglobin in the urine. Uteroglobin could be formulated with a gel or matrix for sustained delivery of a high localized dose, such as at the site of a surgical procedure, particularly vascular surgery to prevent scarring, fibrosis, and reclosing of the arteries. Therefore, one can treat various internal organs and tissues, including the lungs, vasculature, muscle, connective tissue, bone, blood cells, stomach, kidneys, pancreas, liver, intestines, colon, heart, spleen, thymus, uterus, prostate, and bladder by administering uteroglobin topically to the lungs through a drink, pill, nutraceutical, or suppository.

Examples of compositions of the invention include edible compositions for oral administration such as solid or liquid formulations, for instance, capsules, tablets, pills, and the like liquid preparations for orifice, e.g., oral, nasal, anal, vaginal etc., formulation such as suspensions, syrups or elixirs; and, preparations for parenteral, subcutaneous, intradermal, intramuscular or intravenous administration (e.g., injectable administration), such as sterile suspensions or emulsions. However, the active ingredient in the compositions may complex with proteins such that when administered into the bloodstream, clotting may occur due to precipitation of blood proteins; and, the skilled artisan should take this into account.

In such compositions uteroglobin may be in admixture with a suitable carrier, diluent, or excipient such as sterile water, physiological saline, glucose, DMSO, ethanol, or the like. Uteroglobin could be provided in lyophilized form for reconstituting, for instance, in isotonic aqueous, saline, glucose, or DMSO buffer. In certain saline solutions, some precipitation of recombinant human uteroglobin has been observed; and this observation may be employed as a means to isolate inventive compounds, e.g., by a “salting out” procedure.

Further, the invention also comprehends a kit wherein uteroglobin is provided. The kit can include a separate container containing a suitable carrier, diluent or excipient. The kit can include an additional agent which reduces or alleviates the ill effects of the above-identified conditions for co- or sequential-administration. The additional agent(s) can be provided in separate container(s) or in admixture with uteroglobin. Additionally, the kit can include instructions for mixing or combining ingredients and/or administration.

### EXAMPLES

The invention will now be further described with reference to the following non-limiting examples. Parts and percentages are by weight unless otherwise stated.

### Example 1

Recombinant human uteroglobin was administered to several mammalian species via several routes of administration to determine the safety and biological activity of the protein. The protein was given to rats in order to assess pharmacokinetics, bioavailability, and tissue distribution when administered intravenously, intranasally, and by stomach gavage. It was also given intratracheally to very young animals of three large animal species, including premature baboons, premature lambs and newborn piglets. The biological activity of recombinant human uteroglobin and its effect on various aspects of lung function was evaluated in these animal studies. The concentrations of recombinant human uteroglobin in all species were determined using an ELISA assay that is specific for human uteroglobin.

a. Purification of Recombinant Human Uteroglobin

Recombinant human uteroglobin was cloned and expressed by a method similar to that described in copending U.S. application Ser. No. 08/864,357. Protein was purified by proprietary method under FDA guidelines for use as a pharmaceutical agent.

In the alternative, the protein was extracted from the E. coli cell paste by high pressure shear and clarified by centrifugation. The first crude fraction was separated by tangential flow filtration at 100,000 daltons, followed by chromatography on anion exchange and hydroxyapatite supports. The purified protein was further purified by tangential flow filtration at 30,000 daltons.

b. ELISA for Uteroglobin

A quantitative competitive ELISA was developed that provides the necessary sensitivity and precision required for measurement of recombinant human uteroglobin in human samples for use in clinical trials. The assay utilizes a rabbit polyclonal anti-human urine protein-1 (uteroglobin) antibody obtained from Dako, USA. Coating and blocking conditions, antibody dilution, and the type of microtiter plate have been optimized for signal reproducibility and for stability in storage.

Antibody (supplied as a 2 mg/ml stock solution) was used to coat the wells of microtiter plates at a 1:2,500 dilution in a 0.1M carbonate/bicarbonate buffer, pH 9.5. Pipetting of all reagents into microtiter wells was done with an 8- or 12-multichannel pipetter. The diluted antibody solution was pipetted into the wells of Nunc Maxisorp strip plates (100 microliters/well), then the plate strips was sealed in Ziploc bags, and incubated overnight at room temperature (18-25° C.). The next day, the coating solution was removed from each well by aspiration and 200 microliters of blocking buffer (5% sucrose, 5% Bovine serum albumin in phosphate buffered saline) was added to each well. Plate strips were placed back into Ziploc bag and onto a benchtop rotator and rotated gently for 2 hours at room temperature. After the two hour blocking step, the contents of all wells were aspirated and the plate strips were placed upside down in a biosafety cabinet with the fan on, to dry for two hours. Dry plates were stored at 4° C. until ready to use for the assay. Plates prepared in this manner are stable for at least ten weeks.

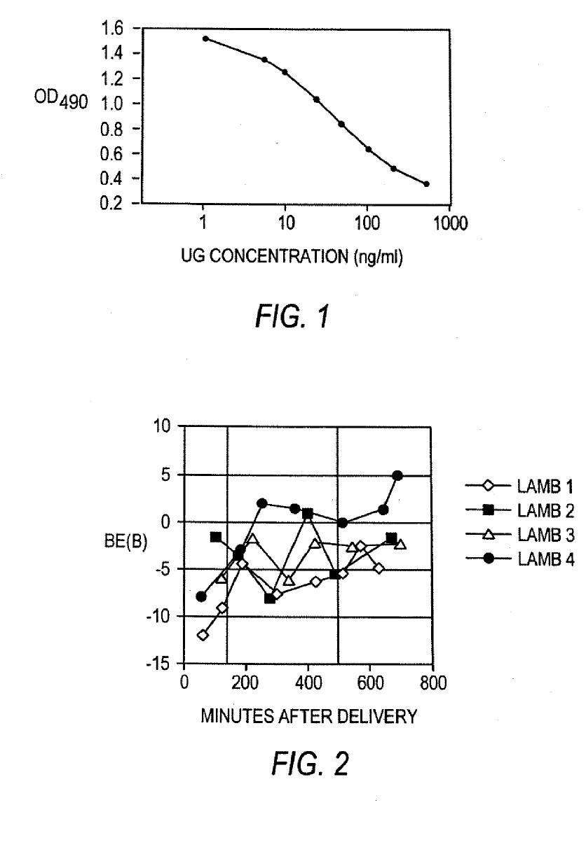

In this competitive immunoassay format, the anti-uteroglobin antibody is used as the capture reagent for any uteroglobin in the sample. A conjugate of horse radish peroxidase (HRP) to recombinant human uteroglobin was generated using a Pierce HRP-labelling kit. The uteroglobin-HRP conjugate bound to the anti-uteroglobin antibody coating the wells, and generated a signal (A₄₉₀) proportional to the amount of HRP-uteroglobin conjugate bound. Signal was generated using a standard HRP enzymatic colorimetric reaction (Pierce OPD substrate). An optimized amount of the uteroglobin-HRP conjugate was mixed with a sample to be assayed (which may be pre-diluted with PBS, if necessary). Typically, two different dilutions (1:2-3 and 1:10) of each sample were run in duplicate, requiring ˜100 microliters of sample. The assay thus revealed a decrease in signal as the uteroglobin in the sample competes with the uteroglobin-HRP conjugate for antibody binding sites, as shown in FIG. 1. A standard curve, using carefully quantitated recombinant human uteroglobin calibrators, was always run in duplicate with each set of samples to assess reproducibility and quantitate uteroglobin in samples.

The uteroglobin ELISA involves a single antibody binding step to capture uteroglobin antigens in each sample. Therefore, the sample must be pre-mixed with the uteroglobin-HRP in an untreated microtiter dish, prior to addition to antibody-coated wells. A 110 microliter volume of recombinant human uteroglobin calibrator, pre-diluted sample or control (PBS only) was mixed with 110 microliter of the uteroglobin-HRP conjugate (1:200 dilution in PBS of 1 mg/ml stock solution) in appropriate wells of a pre-labeled microtiter dish). Once the samples were prepared, the entire 220 microliters was transferred to labelled antibody-coated wells. The microtiter plate strips were incubated at room temperature for 60-75 minutes on a benchtop rotator to allow uteroglobin antigen to bind to the wells. Plates were then washed three times using a microplate washer (Biotek Instruments model) with 0.05% Tween-20 in PBS. The HRP substrate (Pierce OPD) was prepared according to the manufacturer's instructions. Substrate (100 microliters) was then added to all test wells using a multi-channel pipetter and the plate was incubated for 30 minutes at room temperature. Color development was stopped at 30 minutes by adding 50 microliters of 1.2N sulfuric acid to each well. Absorbance was read in a microtiter plate reader (Biotek Instruments EL800) at a wavelength of 490 nm.

A uteroglobin standard curve, uteroglobin sample concentration and test statistics were generated with KC4 software (Biotek Instruments) through a direct interface were between the microplate reader and PC computer. Coefficients of variation (CV) in this assay between less than 8% at concentrations down to 25 ng/ml and were generally lower than 15% between concentrations of 1-25 ng/ml with recombinant human uteroglobin calibrators and in human sera.

c. Intratracheal Administration of Recombinant Human Uteroglobin to Pre-Term Lambs

This study used neonatal lambs delivered by cesarean section as a model of surfactant-dependent neonatal respiratory distress syndrome (RDS). The primary objective of the study was to determine whether the administration of recombinant human uteroglobin would in any way decrease the benefit of surfactant administration in these animals. Since uteroglobin is known to bind to phospholipids, it could thereby interfere with the action of surfactant, which is mainly composed of phospholipids. The severity of RDS in the animals required very aggressive ventilation by human clinical standards, and thus provided a sensitive indication of any effects of recombinant human uteroglobin on the action of surfactant in RDS rescue.

Four lambs were delivered between 135-13& days of gestation by cesarean section and were in respiratory distress at birth. One dose of surfactant was administered after each animal was stabilized on ventilation, and 15 minutes later approximately 5 mg/kg recombinant human uteroglobin (in two animals) or control vehicle (in the remaining two animals) was administered.

Although all animals were of similar gestational age, body weight ranged from 2.6 to 3.6 kg, and condition at birth ranged from mild to severe respiratory distress. The three lowest weight animals (# 1, #2 and #4) had severe or very severe RDS, while animal #3 had milder RDS as judged from the initial chest x-ray. This was also reflected in the pre-treatment blood oxygenation (paO₂), which was considerably better in animal #3 than in the other three animals. Animal #4 was exceptional in that it was born with very severe meconium aspiration. The amniotic fluid was a thick brown consistency and tracheal aspirates from this animal were also brown and very viscous. There were no apparent physical defects but the animal was not active prior to sedation. Chest X-ray showed that the animal had the most severe respiratory distress, with mostly opaque lungs. The two animals treated with recombinant human uteroglobin had the most severe RDS, despite the fact that one of them had severe meconium aspiration with mostly opaque lungs.

All animals received the same surfactant treatment, except that animal #1 received only one dose of surfactant (shortly after birth), whereas the others received two (the first shortly after birth and the second six hours later). Animal #1 required very high ventilator pressure (PIP≧50) to maintain blood oxygenation and developed pneumothorax (confirmed by chest X-ray) at 10.5 hours after delivery. When final fluid samples were taken the animal was euthanized. After treatment of this animal, the protocol was altered to provide for a second dose of surfactant and recombinant human uteroglobin six hours after the first doses in accordance with prescribing information for Survanta. The treated animals were #2 (5 mg/kg×2) and #4 (6.25 mg/kg×2). Animals #1 and #3 did not receive the study drug; animal #1 received no treatment and animal #3 received a volume of normal saline equal to the volume of recombinant human uteroglobin solution given to the treated animals (See Table 1 below).

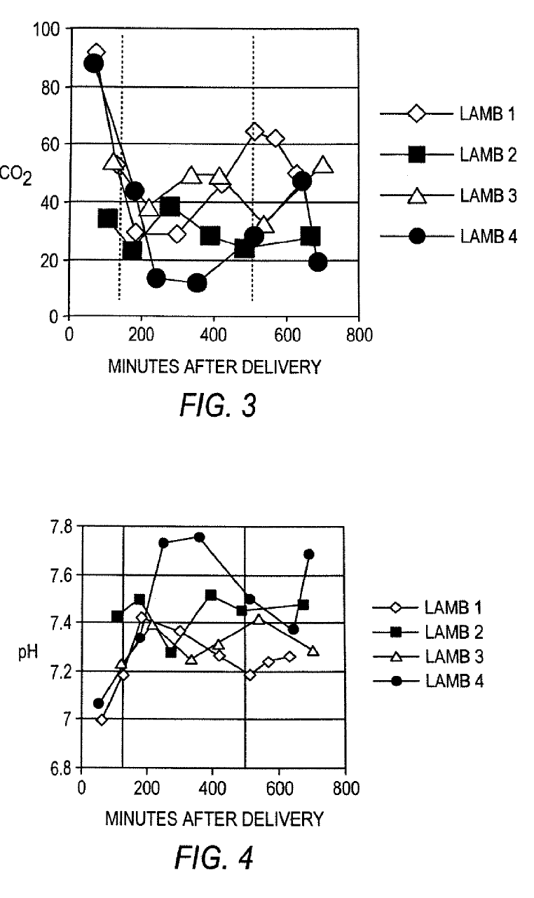

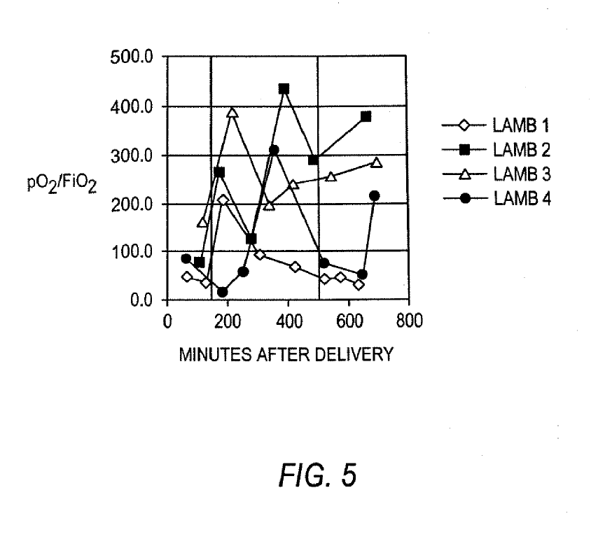

All four animals responded as anticipated to surfactant with increases in paO₂/FiO₂, decreases in paCO₂ and increases in blood pH and bicarbonate excess, BE(B). These data are shown in the FIGS. 2-5 representing blood values as a function of time in each animal. The animal (#2) treated with recombinant human uteroglobin (but without meconium aspiration) showed the greatest response to surfactant administration based on blood gases, even though it had more severe respiratory distress than the animals not treated with recombinant human uteroglobin. The other animal treated with recombinant human uteroglobin, which had meconium aspiration (#4), had significantly delayed responses in paO₂ relative to the other animals (about 180 minutes versus 15-30 minutes), but eventually responded better than the untreated animals. The two treated lambs also developed a higher blood pH relative to the untreated animals, and were the only animals in the study to reach a positive bicarbonate excess. None of the animals received electrolytes or any fluids that might have resulted in induction of a metabolic alkalosis, so the increases in blood pH and bicarbonate excess were due solely to improvements in pulmonary function mediated by recombinant human uteroglobin.

TABLE 1
Summary of Animals Treated

| | Animal #1 | Animal #2 | Animal #3 | Animal #4 |
|---|---|---|---|---|
| Single/twin | Single | Twin (with #3) | Twin (with #2) | Twin (other dead) |
| Sex | Male | female | Female | Female |
| Gest. Age (days) | 134 | 136 | 136 | 135 |
| Weight (kg) | 3.1 | 2.7 | 3.6 | 2.6 |
| Condition at birth | Vigorous Severe | Active Severe RDS | Active Mild RDS | Not active Severe |
| | RDS | | | RDS |
| Amniotic fluid | Clear | Clear | Clear | Meconium |
| Pre-treatment Pa₀₂ | 50 | 80 | 160 | 90 |
| (Torr) | | | | |
| Pre-treatment chest x- | Significant opacity | Significant opacity | Mostly clear | Mostly opaque |
| ray | Poor inflation | Poor inflation | Good Inflation | Poor Inflation |
| Survanta @ birth | 200 mg | 200 mg | 200 mg | 200 mg |
| | (65 mg/kg) | (74 mg/kg) | (55 mg/kg) | (77 mg/kg) |
| @6 hrs | Not treated | 200 mg | 200 mg | 200 mg |
| | | (74 mg/kg) | (55 mg/kg) | (77 mg/kg) |
| Recombinant human | Not treated | 6.25 mg/kg | 0 mg/kg (saline) | 5.0 mg/kg |
| uteroglobin *@ birth | | | | |
| @ 6 hrs | Not treated | 6.25 mg/kg | 0 mg/kg (saline) | 5.0 mg/kg |
| Post-treatment Pa₀₂ | 200 @ 120 min. | 280 @ 100 min. | 380 @ 100 min. | 450 @ 300 min. |
| (Torr) | | | | |
| Survival | 11 hrs | 12 hrs | 12 hrs | 12 hrs |
| Cause of death | Pneumothorax | Euthanized | Euthanized | Ethuanized |

It is clear from this data that recombinant human uteroglobin did not interfere with surfactant replacement therapy and is thus safe to use during surfactant rescue therapy. The animal with severe meconium aspiration responded remarkably well with respect to blood oxygenation and pH, indicators of improved lung function, in the presence of recombinant human uteroglobin. Meconium is known to inactivate surfactant and this animal did not respond to the exogenous surfactant in the normal timeframe of 15-30 minutes, as did the other animals. Therefore, the delayed blood gas responses that each occurred about 180 minutes after each administration of surfactant and recombinant human uteroglobin can be attributed to an independent action of recombinant human uteroglobin on the lung tissue. This is the first observation of the direct effect of uteroglobin on lung tissue.

Lambs were treated and monitored for twelve hours post-delivery. Bodily fluid samples were taken from each animal for analysis of uteroglobin concentration by ELISA as listed in Table 2 below.

TABLE 2
Samples for Uteroglobin Pharmacokinetics

| | Serum | Plasma | Tracheal Aspirate | Urine |
|---|---|---|---|---|
| Pre-Treatment | X | X | | X |
| 2 Hours | | | X | |
| 6 Hours | X | X | | X |
| 12 Hours | | | X | X |

At death, necropsy tissue specimens focusing on lungs were examined for gross pathology and preserved in formalin for histopathological analysis. Animals treated with recombinant human uteroglobin showed no evidence of drug-related toxicity during treatment. Tissues and organs isolated from the animals showed no gross or microscopic abnormalities resulting from recombinant uteroglobin administration. Therefore, intratracheal administration of recombinant human uteroglobin in at least two separate doses of up to 6.25 mg/kg was safe and non-toxic.

The concentration of recombinant human uteroglobin in the lamb fluids was quantitated by uteroglobin ELISA and results are shown in Table 3 below. The recombinant protein followed the same pattern of distribution in bodily fluids as the native protein in humans. That is, it was taken up from the site of administration to the extracellular lung fluids into the blood, and was excreted in the urine. Therefore, the recombinant human protein behaves the same way in vivo as does the native protein.

TABLE 3
Recombinant Human Uteroglobin Concentration in Bodily Fluids
(ng/mL)

| Sample | Animal #1 | Animal #2 | Animal #3 | Animal #4 |
|---|---|---|---|---|
| Tracheal Aspirates | | | | |
| Pretreatment | <5 | <5 | <5 | 47 |
| 1-2 Hrs Post-treatment | nd | >68,200 | 10 | 19,400 |
| 2-4 Hrs Post-treatment | <5 | >68,200 | 11 | nd |
| 4-6 Hrs Post-treatment | nd | nd | nd | 11,900 |
| 6-8 Hrs Post-treatment | nd | >68,200 | nd | nd |
| 8-10 Hrs Post-treatment | nd | 13,000 | nd | 58,200 |
| Plasma | | | | |
| Pretreatment | nd | <5 | <5 | <5 |
| 6 Hrs Post-treatment | <5 | 4,090 | <5 | 584 |
| 8 Hrs Post-treatment | nd | nd | nd | 2,932 |
| 12 Hrs Post-treatment | <5 | 3,795 | <5 | 1,964 |
| Serum | | | | |
| Pretreatment | <5 | <5 | <5 | nd |
| 2 Hrs Post-treatment | nd | nd | nd | 723 |
| 6 Hrs Post-treatment | <5 | 3,455 | <5 | 447 |
| 8 Hrs Post-treatment | nd | nd | nd | 1,730 |
| 12 Hrs Post-treatment | <5 | 3,758 | <5 | 1,563 |
| Mother of animal | nd | nd | 5.3 | <5 |
| Urine | | | | |
| Pretreatment | nd | 5.3 | <5 | 8 |
| 6 Hrs Post-treatment | nd | 31 | <5 | nd |
| Post-mortem (12 Hrs) | <5 | 27 | <5 | 121 |

nd = not done

The bioavailability of intratracheal recombinant human uteroglobin was excellent. The recombinant human uteroglobin-treated animals showed 12-60 μg/mL recombinant human uteroglobin in tracheal aspirates at two hours after the first recombinant human uteroglobin administration, 0.5-4.0 μg/mL in serum and plasma that peaked at four hours after the first recombinant human uteroglobin administration, and 5-100 ng/mL in urine at ten hours after the first dose of recombinant human uteroglobin. This demonstrates that recombinant human uteroglobin can be administered systemically via the lungs, for the purpose of raising circulating levels of the protein, for the purpose of delivering recombinant human uteroglobin to tissues and organs, and for the purpose of raising the concentration of uteroglobin in the urine. Thus, it is possible to treat various internal organs and tissues, including the vasculature, muscle, connective tissue, bone, blood cells, stomach, kidneys, pancreas, liver, intestines, colon, heart, spleen, thymus, ureters and bladder, etc. by administering uteroglobin topically to the lungs by intratracheal deposition or through an inhaler device or nebulizer.

d. Intratracheal Administration of Recombinant Human Uteroglobin to Newborn Piglets Ventilated for 48 Hours

The ventilated newborn piglet was selected for this study because it is a well-characterized model of neonatal lung injury. While the newborn piglet is not a surfactant dependent model, it is an excellent model for neonatal lung injury mediated by oxygen toxicity arising from the use of positive pressure ventilation and elevated oxygen delivery in RDS rescue. Significant decreases in pulmonary compliance, as well as increases in inflammatory markers, indicative of pulmonary inflammation, are observed within 48 hours in this model. Although the model is not surfactant dependent, it is quite responsive to the administration of exogenous surfactant. Thus, the linkage between increased pulmonary inflammation and decreased pulmonary mechanical function that occurs in human neonates who develop chronic lung disease is preserved in this model. Further, direct injury to pulmonary surfactant is measured by analyzing the surface tension properties of surfactant collected by BAL from the lungs of treated and untreated animals. Therefore, this model is well suited to the evaluation of the effect of uteroglobin on lung function and safety of intratracheal recombinant human uteroglobin for the treatment of RDS and the prevention of chronic lung disease in ventilated human neonates.

In this study, piglets were treated with combinations of artificial surfactant and recombinant human uteroglobin or control vehicle and then ventilated with either room air or 100% oxygen for 48 hours. Exogenous surfactant consisted of Survanta (Ross Labs) given intratracheally, in a single dose, via the endotracheal tube and at the recommended human dosage of 100 mg/kg. Recombinant human uteroglobin, formulated in sterile saline (0.9%), was given in a single intratracheal dose, within 30 minutes following the surfactant and at concentrations of 1, 5, and 25 mg/kg. Control groups received comparable volumes of sterile saline only.

A total of 55 newborn piglets were sedated and ventilated for 48 hours with either room air or 100% oxygen, according to Davis, et al. (1993) as shown in Table 4. Twenty-one piglets were ventilated with room air and thirty-four were ventilated with 100% oxygen. Human neonates in respiratory distress always receive supplemental oxygen but it is often less than 100%, depending upon the degree of severity of the RDS and the medical practitioners' individual approach to ventilation management. Therefore, the use of room air and 100% oxygen allows the comparison of treatment extremes in this safety evaluation.

TABLE 4
Groups in the 48 Hour Study

| Recombinant human | Ventilated | Ventilated |
|---|---|---|
| uteroglobin dose group | with Room Air | with 100% Oxygen |
| Control | N = 4 | N = 4 |
| Control vehicle + Survanta | N = 5 | N = 5 |
| 1 mg/kg recombinant human | N = 4 | N = 6 |
| uteroglobin + Survanta | | |
| 5 mg/kg recombinant human | N = 4 | N = 12 |
| uteroglobin + Survanta | | |
| 25 mg/kg recombinant human | N = 4 | N = 7 |
| uteroglobin + Survanta | | |

i. Pharmacokinetics of Recombinant Human Uteroglobin in Newborn Piglets

In humans, a considerable body of evidence indicates that endogenous uteroglobin is produced in the pulmonary and tracheal epithelia, enters the blood by an unknown mechanism, and is eliminated from the blood via the kidney. Three types of fluid samples were collected in order to monitor the half-life and elimination of recombinant human uteroglobin following intratracheal administration. The first type was broncho-alveolar lavage fluid (BAL) collected at 48 hours, after sacrifice, lung excision and pulmonary function testing. This is as close to the site of administration as was practical to sample. The second type was serum collected before treatment and at 2, 4, 8, 12, 24, 36, and 48 hours after administration, from which an estimate of circulating half-life can be made. The third type of sample was urine, collected on the same schedule as serum. Overall, the distribution of recombinant human uteroglobin in piglet fluid was consistent with the distribution of endogenous uteroglobin in humans.

The concentration of recombinant human uteroglobin was measured in BAL, sera, and urine using the competitive ELISA described above. Only three sets of sera from seven untreated piglets were tested in order to establish the background level for the uteroglobin ELISA. The four remaining sets were not tested in the interests of conserving valuable ELISA reagents. Likewise, not all urine samples from untreated piglets were tested. Background immunoreactivity for the uteroglobin ELISA in serum ranged from undetectable to about 100 nanograms/ml and the highest background level in urine was 26 nanograms/ml.

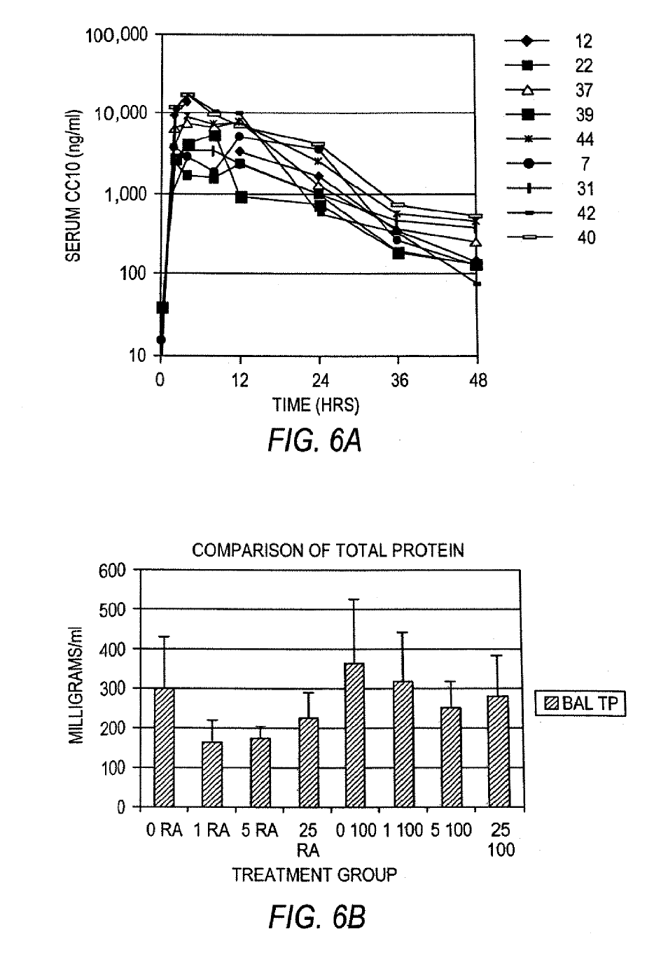

The serum data were fairly consistent among the eight piglets dosed with recombinant human uteroglobin. Recombinant human uteroglobin was detected in all animals at the two hour timepoint. The recombinant human uteroglobin level peaked between two and eight hours post-administration. This shows that either the distribution of the drug in the lungs was variable or that the ability of the lungs to convey recombinant human uteroglobin to the blood was variable, or both. Peak serum levels of 4-17 μg/mL (mean 8.6±6.1 μg/mL) were measured within 2-8 hours of drug administration. Elimination of recombinant human uteroglobin from the serum corresponded well to first-order kinetics between 8 and 48 hours after drug administration (R²=0.97) with a half-life of 7.9 hours. The half-life of uteroglobin in humans has not yet been accurately assessed. At 48 hours, serum uteroglobin levels were still elevated (0.24±0.16 μg/mL) relative to control animals, in which uteroglobin was usually less than the assay detection limit of approximately 0.01 μg/mL Data showing the concentration of recombinant human uteroglobin in serum as a function of time after intratracheal administration are shown in FIG. 6A.

A comparison of the urine recombinant human uteroglobin concentrations in the treated animals in the 100% oxygen group versus the room air groups shows that there may be a difference in the renal handling of circulating recombinant human uteroglobin. Animals #7, 31 and 42 seemed to excrete a much greater amount of recombinant human uteroglobin in their urine during the study period than did animals #12, 39, and 44. Piglet #22 appears to have been dehydrated since it did not produce much urine during the study period, was noted to have bloating and diarrhea within the first twelve hours of the study period, and the investigators noted difficulties extracting blood samples from the animal. It is not known whether this apparent difference in the renal handling of recombinant human uteroglobin reflects a difference in the molecular form of recombinant human uteroglobin or a difference in the kidneys' ability to process recombinant human uteroglobin. The kidney is known to respond to the level of oxygen in the blood and is part of the homeostatic regulatory system, with potential feedback mechanisms to the lungs. An example of an altered molecular form of recombinant human uteroglobin that affects renal handling may involve complexing to a high molecular weight protein like fibronectin, which does not pass through the renal glomeruli. When homodimeric uteroglobin passes through the glomeruli, it is thought to be reabsorbed by the tubules, such that there is approximately a twenty to one hundred-fold steady state difference between the circulating concentration and the urine concentration in normal humans.

A considerable quantity of recombinant human uteroglobin also remained in the BAL fluid after 48 hours. The background level in the BAL of untreated piglets was about 200 nanograms/ml. This relatively high level of background was probably due to cross-reactivity with endogenous porcine uteroglobin or some other protein(s), as well as the matrix effects of this particular fluid in the uteroglobin ELISA. The BAL concentrations in treated piglets ranged from background levels of about 200 nanograms/ml (piglet #42) to over 16 micrograms/ml (piglet #31). The concentration of uteroglobin immunoreactivity in BAL from normal adult humans was 3-8 micrograms/ml with occasional concentrations up to 25 micrograms/ml in asymptomatic normal adults.

It is clear that a significant amount of recombinant human uteroglobin, remains in the extracellular lung fluids for an extended period of time (i.e. two days). In particular, piglets #31 and #44 had significantly higher recombinant human uteroglobin in BAL than the rest of the treated piglets. The reason for this is not understood but there is no indication from either the lung pathology or the pulmonary function tests that these animals suffered any toxic effects as a result of high recombinant human uteroglobin immunoreactivity remaining in their lung fluids. These results also indicate that recombinant form of human uterglobin is most likely utilized and processed by the same pathways as the endogenous protein in the lungs, kidneys and circulatory system, based on parallels with published adult human data. Further, there is no indication that there is a significant difference between the pharmacokinetics of recombinant human uteroglobin in the newborn piglet versus that of endogenous uteroglobin in the adult human.

ii. Total Protein in BAL

A significant indicator of lung injury is the concentration of total protein in lung lavage fluids. Serum proteins are known to leak into extracellular lung fluids when the alveolar-capillary barrier is impaired and vascular permeability is increased. Total protein concentration in BAL samples was measured in each of the animals. The data are shown in FIG. 6B. A major characteristic of this piglet model of oxygen-induced lung injury is nicely illustrated in that the room air (RA) groups all have lower protein content, and less injury, than the groups that received 100% oxygen. There was also a significant difference in total protein in BAL between the treatment groups receiving surfactant with and without rhUG. The groups that received rhUG all contained lower mean protein concentrations, indicating lower levels of lung injury and lower vascular permeability. Thus, rhUG had a significant effect on regulation of vascular permeability, perhaps as a result of interactions with the smooth muscle component of the vasculature, or perhaps as result of an effect on the nitric oxide signaling pathway. This observation was consistent with the apparent benefit of rhUG in pulmonary function tests, in that rhUG-treated animals exhibited better lung compliance that their untreated counterparts.

iii. Pulmonary Function Testing

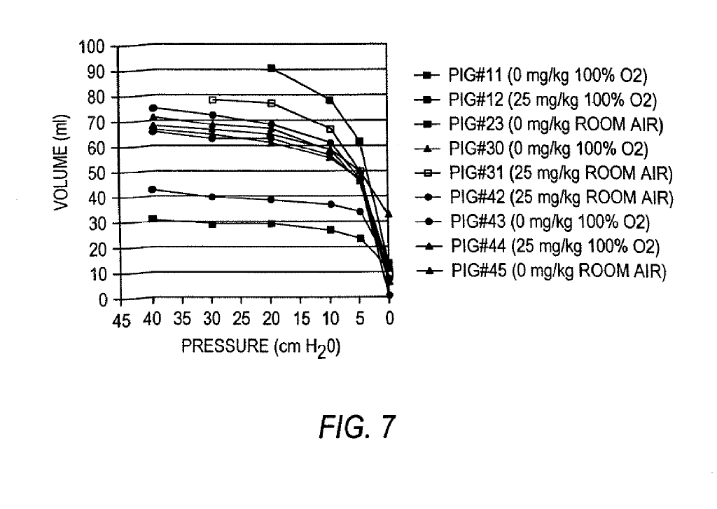

Pulmonary function testing was successfully performed on two thirds of the animals studied, including at least three animals in each of the ten groups. It was apparent after completing the 0 and 25 mg/kg groups that CC10 conferred a benefit. The data are shown in the accompanying figures. FIG. 7 shows the pressure-volume relationships. While most of the animals (5) fall into a single main group, the animals with lower pulmonary compliance (piglet #11 and #43) are both in the 0-100 group. Of the two animals (piglet #23 and #31) that are above the main group with better lung compliance, both were ventilated with room air, one received recombinant human uteroglobin and one did not.

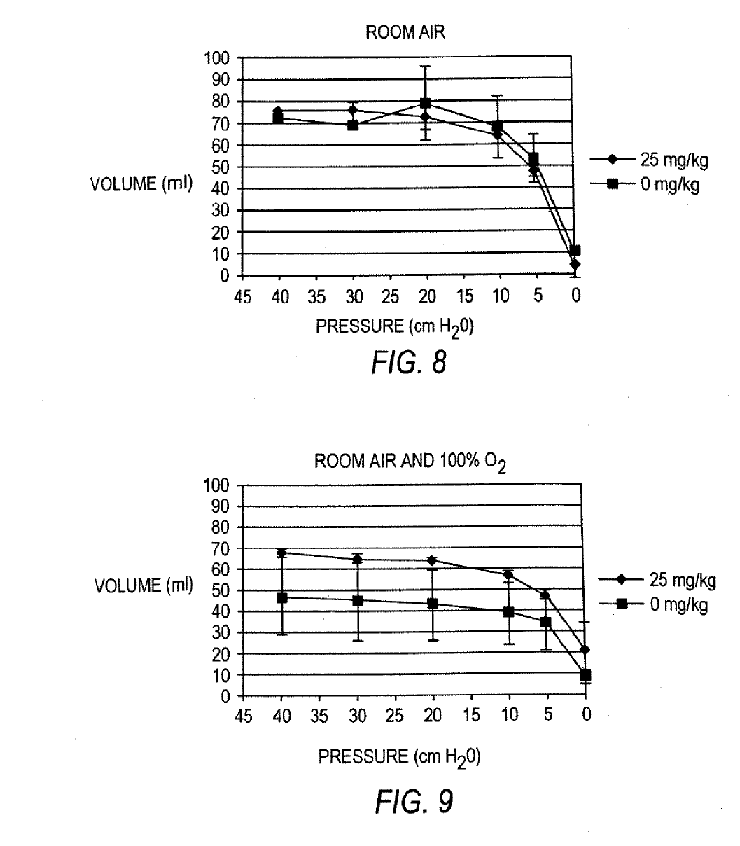

FIG. 8 shows the mean pressure-volume relationships measured for animals ventilated with room air (error bars indicate standard deviation). There was no significant difference in lung compliance between room air-ventilated animals that did and did not receive the study drug (p=0.90).

FIG. 9 shows the mean pressure-volume relationships measured for all animals ventilated with room air and 100% O₂ (error bars indicate standard deviation). There was clearly an improvement in mean pulmonary compliance in animals that were treated with recombinant human uteroglobin compared to saline controls.

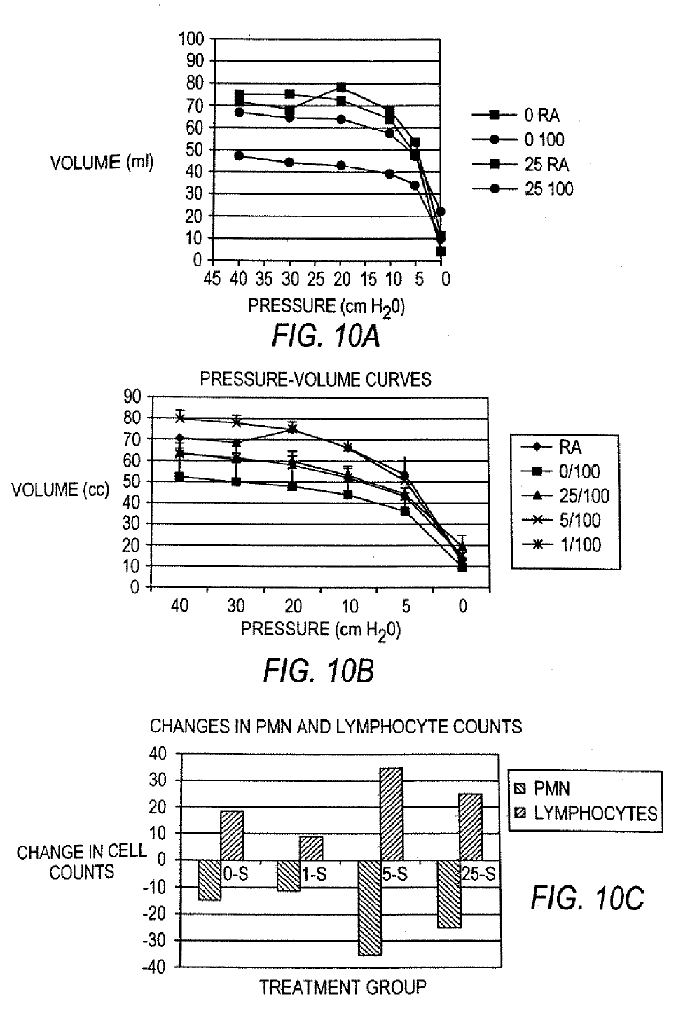

In this model, most of the pulmonary damage manifested as decreased compliance is caused by hyperoxia rather than barotrauma. This effect can be seen in FIG. 10A, in which the mean pulmonary compliance among all four groups is compared. For animals not receiving the study drug, pulmonary compliance was considerably lower in animals ventilated with 100% oxygen (0100) relative to those ventilated with room air (0 RA) (p=0.09). For animals receiving the study drug, however, pulmonary compliance was similar whether or not the animals were ventilated with room air (25 RA) or 100% oxygen (25 100). Thus, recombinant human uteroglobin has countered the negative effects of 100% oxygen on pulmonary compliance.

Further analysis of the 1 mg/kg and 5 mg/kg rhUG dose groups showed that rhUG produced a marked improvement in pulmonary compliance, elasticity and distensibility in groups injured by ventilation with 100% oxygen. These results are illustrated in FIG. 10B. The most effective dose was 5 mg/kg, but 1 mg/kg and 25 mg/kg were also beneficial. This is an extremely important finding in terms of the clinical development of rhUG. We have uncovered a previously unsuspected property of rhUG in relaxing smooth muscle in the lung tissue and vasculature, in the presence of ventilation with oxygen. Inspired elevated oxygen is used clinically in patients with respiratory insufficiency, in acute respiratory distress syndrome, as well as with many chronic lung diseases that result in respiratory insufficiency, including idiopathic pulmonary fibrosis, chronic obstructive lung disease, cystic fibrosis, and other lung diseases. Our results indicate that rhUG can be used to alleviate breathing difficulties in patients requiring elevated oxygen inspiration, and may offset or reduce the pulmonary toxicity due to the oxygen itself.

e. Long Term Effects of Intratracheal Administration of Recombinant Human Uteroglobin to Newborn Piglets

Twenty newborn piglets were sedated, entubated and dosed according to the groupings shown in the Table 5 below. They were then allowed to recover and were maintained for one month. (Animals were bottle-fed for the first two weeks.) After 28 days the animals were sacrificed and necropsied for a full toxicological evaluation.

Several analyses were performed on piglets samples as shown in the Table 6. No evidence of toxicity was observed, even at the highest dose of recombinant human uteroglobin. There was no evidence of an anti-recombinant human uteroglobin antibody production at 28 days. Recombinant human uteroglobin did not persist in the circulation of the animals for 28 days, consistent with the 7.9 hour half-life measured in the 48 hour study. There was no long-term toxicity observed for intratracheal recombinant human uteroglobin administration in the newborn piglet.

TABLE 5
28 Day Piglet Study Groups

| | Recombinant human | |
|---|---|---|
| Number of Piglets | uteroglobin dose | Survanta dose |
| 4 | 0 mg/kg | 0 mg/kg |
| 4 | 0 mg/kg | 100 mg/kg |
| 4 | 1 mg/kg | 100 mg/kg |
| 4 | 5 mg/kg | 100 mg/kg |
| 4 | 25 mg/kg | 100 mg/kg |

TABLE 6
Summary of Samples and Analyses for 28 Day Piglet Study

| Data or Sample Type | Type of Analysis | Pre-Rx | 28 Days |
|---|---|---|---|
| Whole blood | CBC & Differentials | X | X |
| Serum | uteroglobin ELISA | X | X |
| Serum | Anti-uteroglobin antibody titer | X | X |
| Whole animal weights | Overall growth | X | X |
| Necropsy | Gross pathology | | X |
| Organs in formalin: | Weights and histopathology | | X |
| Lungs (both) | | | |
| Heart | | | |
| Liver | | | |
| Thyroid | | | |
| Adrenals (both) | | | |
| Spleen | | | |
| Kidneys (both) | | | |
| Brain | | | |
| Lymph nodes | | | |

f. Piglet Lymphocyte Data

CBC (complete blood counts) and differential white counts were done in the hospital lab on whole blood collected prior to uteroglobin administration and at 28 days post-administration, immediately prior to sacrifice. The mean cell counts before rhUG treatment and at 28 days were calculated for each group, then the differences between the mean initial and final cell counts were calculated. There was a significant difference in the changes in PMN (mature neutrophils) and lymphocyte (T cells, B cells, NK cells) cell counts between the animals receiving rhUG and the control groups. These data are shown in FIG. 10C. (The control group that did not receive either surfactant or rhUG was comparable to the surfactant only group and is not shown.)

The data show a powerful effect of a single dose of rhUG in the newborn piglets. In the control group, the lymphocyte counts went up and the PMN counts went down over the one month period. This is probably normal in the development of the immune system after birth. The rhUG significantly enhanced both of these changes. At a dose of 5 mg/kg, rhUG nearly doubled the increase in lymphocytes, and more than doubled the decrease in PMN. It is clear that the rhUG significantly enhances lymphocyte proliferation and decreases PMN production over the long term. It has been postulated that a hemopoietic stem cell exists that can be committed to either the myeloid or lymphoid lineage. Our data further indicates that such a stem cell exists and that recombinant human uteroglobin is the differentiation factor that channels the development of stem cell precursors from the PMN lineage (myeloid) to the lymphocyte lineage (lymphoid). This is a powerful pharmacologic effect and there is no known agent with equivalent activity. This discovery enables the application of rhUG to several clinical indications involving long term imbalances between myeloid and lymphoid cell counts, treating patients with an excess of myeloid cells, or a deficiency of functional lymphoid cells. This mechanism, for example, may be important in maintaining immune surveillance, mediated by lymphoid cells, in preventing or treating cancer. It will also be important in controlling conditions characterized by an excess of myeloid cells, such as asthma (an excess of eosinophils), or leukemia.

g. Administration of Recombinant Human Uteroglobin to Wistar Rats

The primary purposes of the animal pharmacology and toxicology models described in the Examples thus far is to support the clinical trials of recombinant human uteroglobin in neonatal lung disease. However, they are also applicable to studies of adult lung disease and provide certain basic information about the distribution, metabolism and excretion of the protein.

Uteroglobin is a naturally occurring mammalian protein for which there are no known post-secretory modifications, with the possible exception of alterations in oxidation state, depending upon whether zero, one or two of the possible disulfide bonds between the monomers of the dimeric protein are present. Therefore, it is very unlikely that biotransformation is a consideration in the pharmacodynamics of the recombinant human uteroglobin drug. The main source of endogenous uteroglobin protein in the body is the lungs and it is eliminated from the circulation by the kidneys. The tissue distribution or kinetics of elimination of recombinant human uteroglobin from the body has not been determined when the recombinant protein drug is administered in doses representing a much higher level than the endogenous uteroglobin circulating in the body. But once the normal, circulating physiological concentration is reached (about 150 nanograms/ml in serum), then the elimination kinetics for recombinant protein drugs can be expected to follow the pattern of the endogenous protein, provided the drug is biologically equivalent to the native protein.

The steady-state level of endogenous uteroglobin in the blood is 80-150 nanograms/ml and 2-50 nanograms/ml in the urine of healthy individuals. Bernard, et al., showed that the kidney mediates the excretion of recombinant human uteroglobin from the blood into the urine by comparing serum and urine concentrations of native uteroglobin in normal healthy humans to those of patients with different types of renal impairment. The glomeruli of the kidney filter out small molecular weight waste products and proteins, with a size cutoff of approximately 40 kilodaltons. Uteroglobin is a very compact globular protein with a Stoke's radius of only 18.4 Angstroms, despite a molecular weight of 16 kilodaltons. Crystallization studies verify the very compact structure of this protein. In addition, uteroglobin will pass through a dialysis membrane with a molecular weight cutoff of 8.0 kilodaltons. Patients with either glomerular or tubular disease had high urine concentrations of uteroglobin, which was comparable to that of the serum in patients with the most severe renal disease, and human albumin injected intravenously in rats was reported to competitively inhibit tubular reabsorption of uteroglobin. Together, these results indicate that the uteroglobin homodimer may pass through the renal glomeruli, but is selectively reabsorbed by the renal tubules prior to urinary excretion. Therefore, a significant proportion, as much as 30%, of the circulating endogenous uteroglobin may be removed by healthy kidneys and is excreted in the urine.

A pharmacokinetic study of recombinant human uteroglobin administration to rats was undertaken to provide basic information on half-life, tissue distribution, metabolism and excretion of the protein. A single dose of radiolabeled recombinant human uteroglobin was administered to a total of 12 Wistar rats by three routes of administration: intravenous, intranasal, and stomach gavage. Fluid samples were taken over a period of 24 hours, after which the animals were euthanized and dissected. Radioactivity was measured in all fluids and tissues.

Adult Wistar rats, ages 8-10 weeks, each received approximately 25 million dpm of highly-purified ¹²⁵I-labeled recombinant human uteroglobin (Lofstrand Labs, Inc.), corresponding to 1.9 μg of recombinant human uteroglobin or 13.6 μCi of ¹²⁵I (specific activity: 7.18 μCi/μg protein). Four rats, two male and two female, were included in each of the three groups that received the ¹²⁵I-labeled recombinant human uteroglobin by the three routes of administration. Each animal received 7.17-8.64 μg/kg of recombinant human uteroglobin, corresponding to 51.3-61.6 μCi/kg of ¹²⁵I. Doses of recombinant human uteroglobin varied slightly between animals due to differences in body weight. Animals were housed in metabolism cages so that urine and feces could be collected for analysis at the end of the 24-hour study period. Blood (200-300 μL) was collected at 1, 2, 4, 8, 12 and 24 hours after administration of the radiolabeled protein, and sera and plasma were prepared. The stomach gavage group were also sampled at 30 minutes after administration. Animals had free access to food and water throughout the study period. After final blood samples were collected, animals were exsanguinated and necropsied. In addition to the blood samples, 25 different tissue samples representing all major organs and tissues were collected. Samples were frozen at −80° C. to preserve them for radiation measurements and protein extraction and analysis. Two animals died during the study, one apparently due to blood loss and the other apparently due to handling trauma. Neither death appeared related to the study drug.

Fluid and tissue samples were counted in a gamma counter to determine the amount of radioactivity present. Ten microliters of each sample of serum, plasma and urine were added to 3 mls of scintillation cocktail and counted for one minute each. The samples are analyzed using the competitive ELISA described above to determine concentrations of uteroglobin antigen in all fluid samples and protein extracts of tissues. Likewise, frozen organs were bisected and the intact half was placed in a scintillation vial containing 3 mls of scintillation cocktail and counted for one minute each.

The remaining half of each organ sample was ground up in a 1.5 ml Eppendorf tube, using a motorized pestle (Fisher brand) and the powdered sample was resuspended in a protein extraction buffer (50 mM Tris-HCl, pH 7.6, 1% Triton X-100, 150 mM NaCl, 2 mM PMSF, 10 mM EDTA, 20 mg/ml each aprotinin, leupeptin, and pepstatin A) and vortexed briefly to mix. The mixture was then centrifuged in a microfuge at 4° C. for 15 minutes. The supernatant was transferred to a clean tube and the pellet was discarded. SDS-PAGE analysis was performed on the supernatants, followed by autoradiogram to analyze the pattern of radioactive protein in the tissue samples.

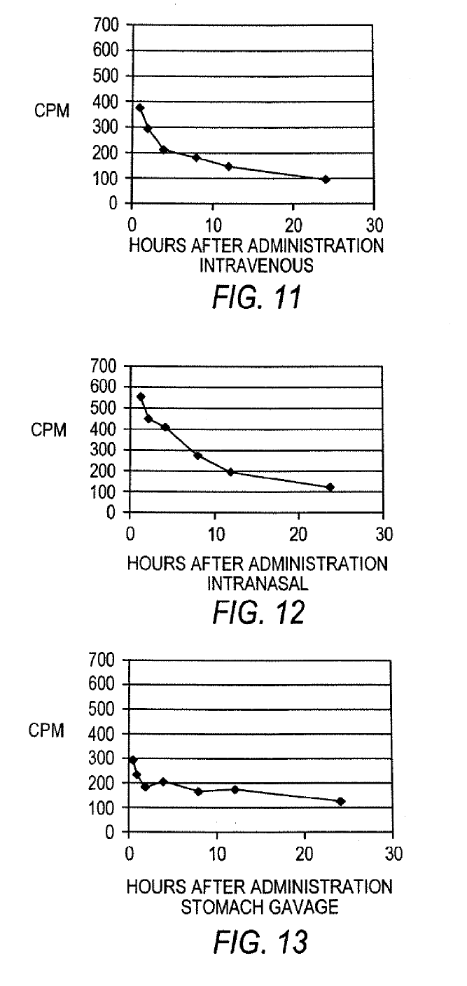

FIGS. 11-13 show radioactive counts as a function of time for each of the three administration groups: intravenous, intranasal, and stomach gavage, respectively. The amount of radioactivity in the blood decreases over time, as expected, and appears to follow first-order elimination kinetics in the intravenous administration group. The elimination patterns in the intranasal and stomach gavage groups are not as distinct as that of the intravenous administration group, presumably due to absorption effects in the sinuses and gastrointestinal tract.

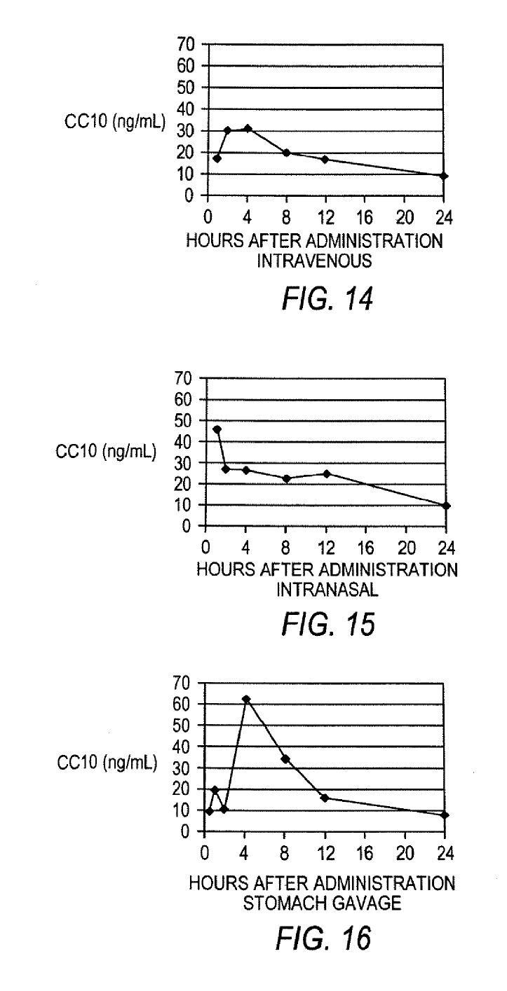

Radiation counts corresponding to recombinant human uteroglobin protein in these samples was confirmed by analyzing them with the uteroglobin ELISA that does not recognize free iodine or degradation products. These data are shown in FIGS. 14-16. In contrast to the radiation counts, there is a delay of 1-4 hours in reaching the peak uteroglobin concentration in the serum for the intranasal and oral routes. These data demonstrate that there is an uptake phase for uteroglobin across the mucosal surfaces of the stomach and upper respiratory tract. It is clear, however, that both the intranasal and oral routes of administration were effective in delivering a significant dose of the protein to the circulation. It was remarkable and unexpected that these routes would be so effective for uteroglobin administration. The nasal mucosa is a barrier between the external flora containing bacteria and viruses, as well as inhaled antigens, and the internal system. Likewise, the gut (via the stomach) is also a selective barrier that distiguishes between food molecules and other ingested materials that are not food. These barriers are generally not permeable to molecules as large as uteroglobin and the specific and efficient uptake shows that there is an active or passive transport system that allows entry of uteroglobin in both the respiratory tract and the digestive tract.

Results in the intranasal group are also consistent with pharmacokinetics observed in both the neonatal lamb and piglet studies, in which uteroglobin persists for an extended period of time in the extracellular lung fluids. This indicates that the extracellular mucosal fluid of the mammalian respiratory tract may serve as a reservoir from which the protein enters the blood. Inhalation may thus be a practical route for systemic administration of the protein in humans. The presence of radioactive recombinant human uteroglobin homodimer in protein extracts of trachea, bronchi, esophagus, and thyroid in an animal from each administration route show that these tissues take up uteroglobin from the circulatory system. This demonstrates that one route may be effective in the specific delivery of protein to target delivery for another system. For example, the intravenous route may be used to deliver a large dose of uteroglobin specifically to the gut via the esophagus or to the lungs via the trachea. Likewise, inhaled uteroglobin may be used to deliver uteroglobin to the gut and kidneys and oral uteroglobin may be used to deliver the protein to the lungs and kidneys. In summary, the data indicate that recombinant human uteroglobin enters the circulation by all three routes of administration tested proving the feasibility of intranasal and oral uteroglobin administration in humans.

### Example 2

### Binding of UG to Fibronectin

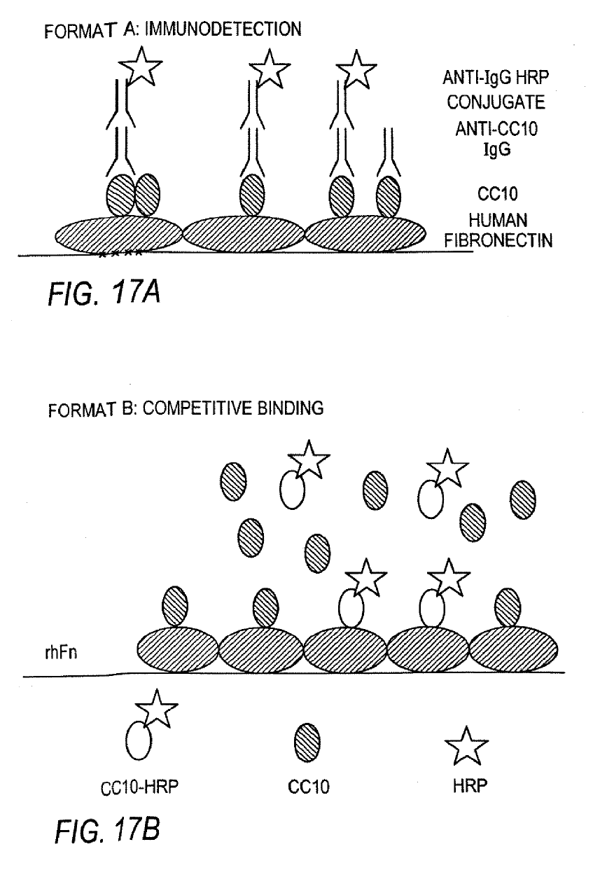

The demonstration of the binding interaction between human fibronectin and recombinant human uteroglobin prompted the development of a non-radioactive assay for this interaction that could be used as a measure of recombinant human uteroglobin biological activity. Therefore, two ELISA-based assay formats for the uteroglobin-fibronectin binding interaction were tested, as shown in FIGS. 17A-17B. Briefly, in the first of these assay methods recombinant human uteroglobin was used to coat the wells of a microtiter dish, which was followed by fibronectin binding and detection of the bound fibronectin with an anti-fibronectin monoclonal antibody (Life Technologies, Inc.; product #12062-014). In the second assay, purified human fibronectin was used to coat the wells of a microtiter dish, followed by recombinant human uteroglobin binding and detection of the bound recombinant human uteroglobin with an anti-uteroglobin antibody (Dako, USA). Both formats gave comparable results with a 2-3 fold signal increase over controls when similar concentrations of recombinant human uteroglobin and hFn were used. There being no apparent advantage of one format over the other, the second format was chosen for further development and analyses due to the relative ease of reagent availability. A detailed description of the second ELISA assay follows.

Purified human fibronectin (hFn) was obtained as a frozen lyophil from Life Technologies, Inc. (Gaithersburg, Md.), product #33016-015. According to the supplier's product specifications, this hFn preparation was purified from human plasma. The hFn was resuspended in sterile water to a concentration of 1 mg/ml. This stock solution was then aliquoted and unused aliquots were frozen at −80° C. An aliquot of the 1 mg/ml stock solution was diluted to 10 micrograms/ml with phosphate buffered saline (PBS) (pH 7.4). Fifty microliters of the diluted hFn were used to coat wells of a microtiter plate (Falcon) with 5 micrograms/well. Further dilutions were made to generate a set of wells containing 1 microgram and 500 nanograms of hFn as well. The hFn coating was performed overnight at room temperature. After coating, the hFn was removed by aspiration and the wells were blocked with a 1:1 dilution of Pierce Blocker™ in PBS (pH 7.4), with a final concentration of 5% BSA in 1×PBS (pH 7.4). The blocking reagent was diluted immediately prior to the addition of 320 microliters per well. The plate was incubated for two hours at room temperature with gentle shaking. The blocking reagent was removed by aspiration and the wells were washed three times with 1×PBS (pH 7.4).

Purified recombinant human uteroglobin, diluted from a 40 mg/ml stock solution in PBS, was added to the wells in a constant volume of 50 microliters but in different concentrations, ranging from 1 ng/well to 1 microgram/well. After the addition of recombinant human uteroglobin or PBS controls, the plates were gently shaken for one hour at room temperature. The recombinant human uteroglobin was then aspirated off and the wells were washed three times with PBS and blocked again for an hour with undiluted Pierce Blocker™ at room temperature. After re-blocking for an hour, the block buffer was removed by aspiration and the wells washed three times with 1×PBS, pH 7.4. Two hundred microliters of a 1:2,000 dilution of a rabbit polyclonal anti-human urine protein-1 (uteroglobin) antibody (Dako, USA) in PBS, were then added to each well. The plate was gently shaken for 1 hour at room temperature, then the antibody solution was removed by aspiration the plate was washed three times with PBS, and blocked again for one hour as described above.

A goat anti-rabbit IgG antibody, conjugated to horse radish peroxidase (HRP), was used to quantitate the amount of anti-uteroglobin antibody in the wells. Two hundred microliters of a 1:20,000 dilution of the HRP-conjugate in PBS was added to each well and the plate was shaken gently for 1 hour at room temperature. The conjugate solution was removed by aspiration and the plate was washed three times with PBS. A HRP substrate (Pierce ODP, made to the manufacturer's instructions) was added (320 microliters) to each well and the colorimetric reaction proceeded for 30 minutes. The HRP reaction was stopped at 30 minutes by pipetting 50 microliters of 1.2N sulfuric acid into each well using a multi-channel pipetter. The absorbance at 490 nm was read in a microtiter plate reader. In order to compare results in separate experiments, data was normalized by expressing the signal generated in uteroglobin-fibronectin test wells as a percent of the signal from fibronectin alone.

Fibronectin is a >200 kDa glycoprotein with three types of repeating elements, called domains, which share a highly conserved secondary structure and a moderately conserved primary amino acid sequence. There are eight type I domains located in the N-terminal third of the human fibronectin protomer and three type I domains at the C-terminus of hFn. There are two type II domains, clustered in the middle of the protomer. There are between 15-17 type III domains in hFn, depending upon the tissue of origin of the fibronectin. In plasma fibronectin, the circulating type produced by Life Technologies Inc. the liver, Type III domains EDA and EDB are not present.

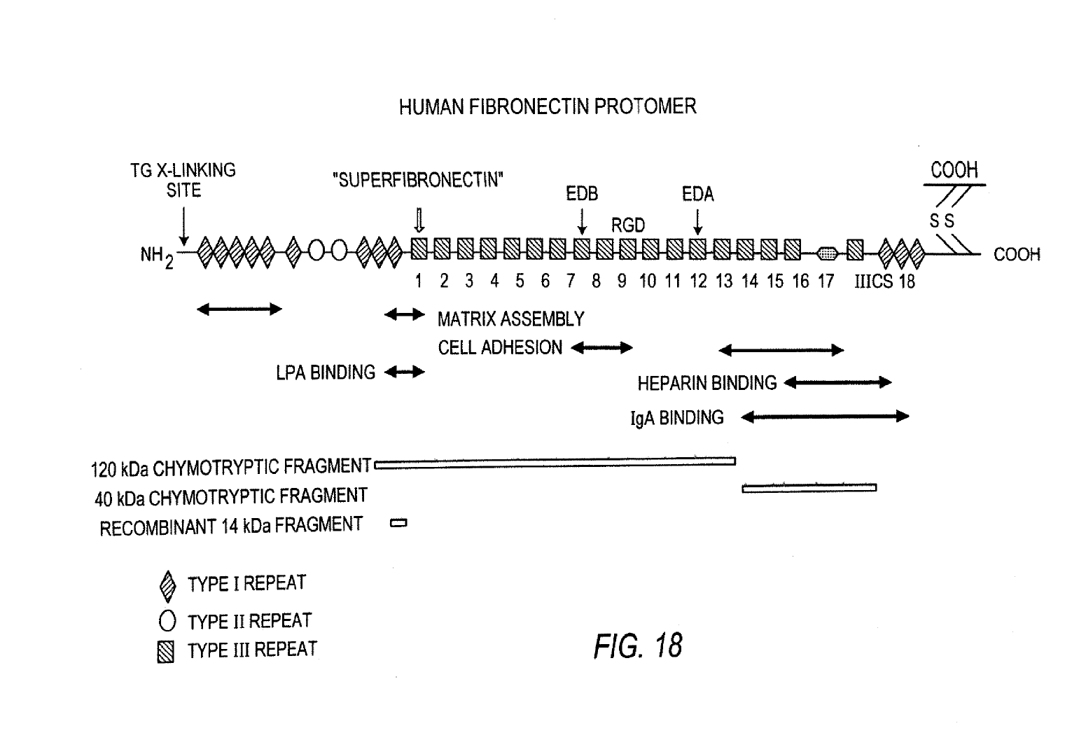

An N-terminal proteolytic fragment of hFn was not available and could not be tested. However, the 120 kDa and 40 kDa chymotryptic fragments of hFn (obtained from Life Technologies Inc.) encompass about 70% of the length of the intact fibronectin molecule, as shown in the map of fibronectin in FIG. 18. Type III domains #1-11 are present in the 120 kDa chymotryptic fragment as shown in the diagram. Type III domains #12-17 are present in the 40 kDa chymotryptic fragment. The clear dose-response curve for the 40 kDa chymotryptic fragment shows that recombinant human uteroglobin binds to at least one other hFn type III domain present in this fragment (#12-17). The recombinant type III domain #1 (referred to as III.1) is found within the 120 kDa chymotryptic fragment.

Reports that recombinant human uteroglobin and human fibronectin form a complex in solution with a measured binding constant of 13 nM and demonstrated biological activity in vivo in mice indicate that an uteroglobin fibronectin complex could exist in vivo in humans. Indeed, high background readings for the purified human fibronectin using the anti-uteroglobin antibody (DAKO) in this uteroglobin-fibronectin binding assay led to speculation that there may be endogenous native uteroglobin that copurified with the fibronectin from human plasma. However, this solution phase interaction occurs at much lower concentrations of uteroglobin and Fn than the interaction seen between recombinant human uteroglobin and insoluble fibronectin. In addition, the solution phase interaction was shown to be relevant to fibronectin polymerization, conversion to the insoluble form, the initial fibronectin deposition in the extracellular matrix or on cells, and in the process of fibrillogenesis. At low concentrations of plated fibronectin and recombinant human uteroglobin no interaction can be detected in this assay. On the basis of this apparent difference, it can be inferred that the downstream process of cell adhesion to the deposited fibronectin during inflammatory cell and fibroblast migration may also be effected by the presence of uteroglobin. Therefore, the following hypotheses were tested in the experiment below: (1) is there a uteroglobin-like antigen in the intact hFn preparation, and (2) does recombinant human uteroglobin binds to portions of fibronectin that are important in cell adhesion and not relevant to fibrillogenesis.

Two commercially available chymotryptic fragments of hFn were selected for experimentation. These fragments were more highly purified than the hFn, having gone through several stages of chromatographic purification after proteolytic cleavage. These chymotryptic fragments are referred to as the 120 kDa fragment and the 40 kDa fragment, both type III domains that are involved in cell adhesion. However, only the 120 kDa fragment contains the region of fibronectin required for polymerization. Fibronectin self-polymerization activity has been localized to an N-terminal 70 kDa proteolytic fragment of human fibronectin, specifically a region including the type I.9-type III.1 domains. The C-terminal 70 amino acid part of the type III.1 domain was produced recombinantly (by Morla et al., 1994) and dubbed “Superfibronectin” because of its tremendous ability to promote fibronectin-fibronectin interactions, polymerization, deposition, and cell adhesion in vitro. Superfibronectin also has the advantage that no endogenous human uteroglobin could possibly be present in the protein preparation because it was purified from bacteria.

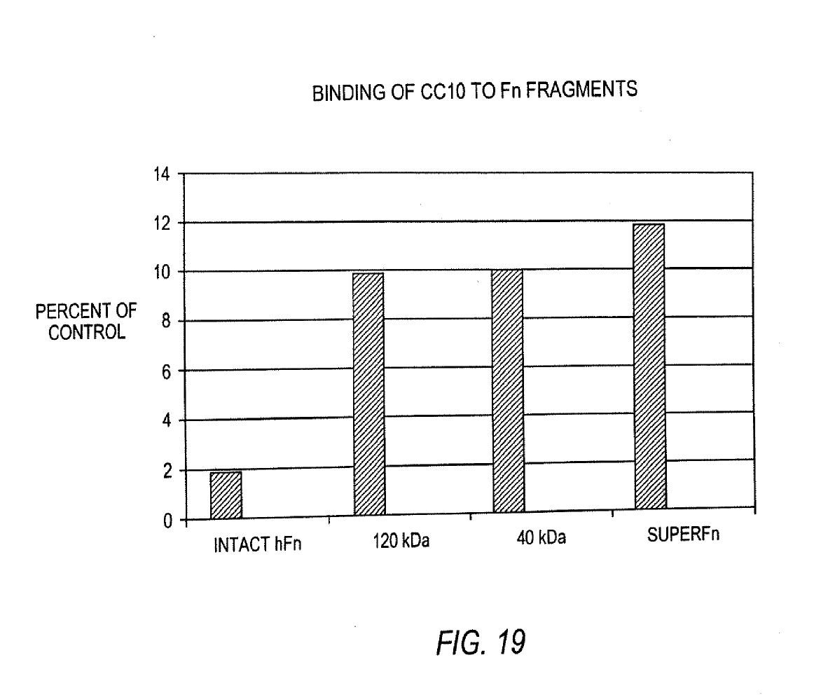

The three pieces of fibronectin were tested in parallel with the intact purified human fibronectin. Five micrograms per well of each Fn species were tested as described above, for binding to 250 nanograms of recombinant human uteroglobin/well. Two such experiments were performed, in duplicate, and the results are shown in FIG. 19.

In the absence of recombinant human uteroglobin the two chymotryptic fragments and the recombinant type III domain, did not show an appreciable signal. Therefore, endogenous uteroglobin is present in the purified intact fibronectin preparation at an approximate concentration of 250 nanograms of uteroglobin per 5 micrograms of hFn. This represents a molar ratio of 17:1, using molecular weights of 16 kDa for the uteroglobin homodimer and 200 kDa for the fibronectin protomer. Second, the binding of the recombinant human uteroglobin to both the 120 kDa and the 40 kDa fragments shows that there is more than one recombinant human uteroglobin binding site in plated fibronectin. The binding of recombinant human uteroglobin to superfibronectin localizes this multiple binding phenomena to the Fn type III repeats, which are present in both the 120 kDa and the 40 kDa fragments.

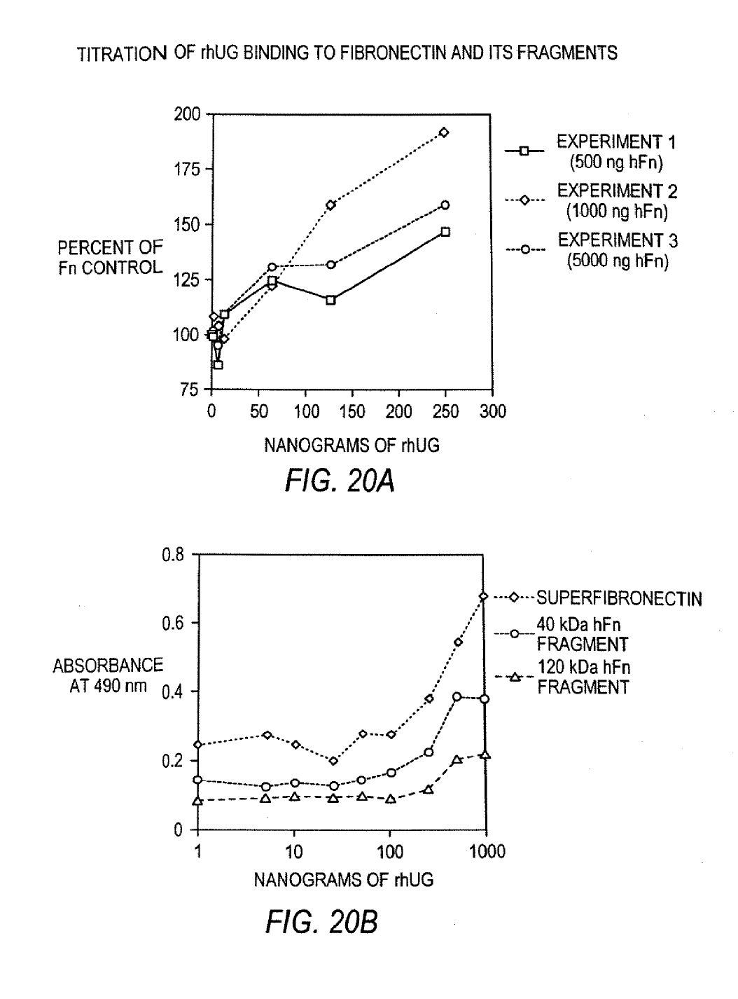

A titration of recombinant human uteroglobin was also done with each fibronectin preparation, in parallel. All four of these preparations were used to coat plates as described, using one microgram of protein per well. Titration curves were done in which various amounts of recombinant human uteroglobin were bound to the intact purified hFn, the two fragments and the recombinant superfibronectin as shown in FIGS. 20A and 20B. This experiment was done in duplicate and the uteroglobin-fibronectin binding step was done in the presence of 2.7 mM calcium chloride, in 1×PBS (pH 7.4) (which is consistent with the normal concentration of calcium in the serum).

There are three conclusions to be drawn from these results: first, the clear dose-response relationship in binding between recombinant human uteroglobin and superfibronectin shows that recombinant human uteroglobin binds to the domain of fibronectin which is also contained in superfibronectin; second, recombinant human uteroglobin binds to more than one Fn Type III domain, because there is a clear dose-response relationship for recombinant human uteroglobin binding to the 40 kDa chymotryptic fragment, which does not contain the superfibronectin peptide sequence, i.e., the type III.1 domain.

The Fn III.10 domain is known as the cell adhesion domain and it contains the only “RGDS” cell adhesion motif present in the entire hFn molecule. Mutagenesis of this sequence to “RGES” severely diminishes adhesion of fibroblasts and pro-inflammatory immune cells, such as neutrophils, to fibronectin. A monoclonal antibody against the cell adhesion domain, clone 3E3, is capable of blocking cell adhesion to fibronectin. Since recombinant human uteroglobin binds to the insoluble cell adhesion domain and to insoluble intact hFn and its chymotryptic fragments, it may be capable of blocking cell adhesion to fibronectin, via an anti-inflammatory, anti-fibrotic and anti-metastatic mechanism.

This hypothesis was tested in cell culture by assaying adhesion of NIH-3T3 cells (ATCC deposit # CRL-6589), an immortalized mouse fibroblast cell line, to hFn-coated wells (BioCoat plates, supplier). The average results from two of experiments are shown in Table 7 below and clearly demonstrate that recombinant human uteroglobin is a potent inhibitor of cellular adhesion to fibronectin.

TABLE 7

| | Recombinant human | | |
|---|---|---|---|
| | uteroglobin | Anti-Fn mAb | Myoglobin |
| | 50 ng | 10 μg | 10 μg |
| % Inhibition | 54% | 60% | 21% |

Clearly, the recombinant human uteroglobin was a potent inhibitor of cellular adhesion to fibronectin. It was nearly as effective as the anti-Fn mAb, but at a 200-fold lower concentration. Myoglobin is a protein that is thought to be irrelevant to the physiology of both uteroglobin and fibronectin and was selected as a non-specific protein control because it is also a circulating protein that is roughly the same size as uteroglobin.

Both the uteroglobin four helical bundle motif and the Fn Type III domain represent fundamental protein structural motifs present in many different proteins and it is reasonable to infer that these motifs share a certain affinity for each other, independent of the context of each individual protein. In fact, all 17 isolated hFn Type III domains mediate cell adhesion to some degree. It, therefore, follows that it is the availability of these domains for binding to other moieties in the surrounding environment that determines whether hFn interacts with other proteins, the extracellular matrix, or with cells. Indeed, the conformation of hFn is known to change from an elongated disk-like globular shape in solution to a stretched-out Y shape upon deposition onto a surface (Erickson & Carrell, 1983). The Fn Type III domains are present in nearly all protein components of the extracellular matrix (e.g., laminin, collagens, vitronectin, fibrin) as well as numerous membrane bound proteins, including adhesion molecules, integrins, and receptors. This Fn type III repeat domain, as well as the four helical bundle, takes on a characteristic secondary structure that is thought to be highly conserved, despite differences in primary amino acid sequence.

Fibronectin Type III repeats are found in a large number of extracellular matrix proteins, as well as in a number of cell surface receptors. Based on their distribution in certain proteins, these domains would seem to play an integral role in cell-cell and cell-extracellular matrix interactions. Our discovery that the four helical bundle of UG and the Fn Type III repeat domains broadly interact enables us to more rapidly identify proteins with these motifs that are likely to interact in physiological and disease processes. As such, this discovery represents a powerful tool for drug discovery and the elucidation of pharmacologic pathways. For example, secretory phospholipases are proteins that contain four helical bundle motifs. Very little is known about how these enzymes act as signalling molecules. While some receptors have been identified, they do not appear to be the receptors through which these enzymes mediate some of their biological effects such as increasing cell proliferation. It is possible to identify novel candidate receptors for these proteins using our discovery, by searching existing publicly-accessible databases such as Genebank. This is done through protein-based similarity searches to uncover new sequences with similarity or partial identity with the query sequence (an FN Type III repeat from fibronectin for example). Alternatively, one can perform a keyword search of the entries for the annotation of Fn Type III repeat or four helical bundle motif. Such candidates would be membrane bound proteins with one or more Fn Type III repeat domains in their extracellular components. Likewise, the converse is also possible. Indeed, we have used this bioinformatics/proteomics approach to identify the membrane bound signalling protein, CD148, containing 8-10 Fn Type III repeats, as a possible receptor for UG. This enables us to directly test whether rhUG interacts with CD148 to mediate some or all of its biological activities.

### Example 3

### Inhibition of Cell Adhesion to Fibronectin by rhUG

The discovery that recombinant human uteroglobin binds to human fibronectin in solution has profound implications (See U.S. Ser. No. 08/864,357). In addition, the ability of recombinant human uteroglobin to prevent fibronectin aggregation in vitro, fibronectin-mediated fibrillogenesis in cell culture, and renal fibronectin deposition in vivo, demonstrates the important physiological role of endogenous uteroglobin in all mammals. Fibronectin is one of the most well characterized mediators of cell adhesion, and is involved in several physiologic processes, including platelet aggregation (thrombosis), wound healing, fibrosis, inflammatory cell and fibroblast adhesion, tumor metastases, and extracellular basement membrane formation. However, these processes involve the insoluble form of fibronectin, not the soluble form. It would be desirable to prevent the conversion of fibronectin from its soluble form to its insoluble form, which could be exploited to prevent the initiation of the processes listed above, or in limiting the extent of such processes. It has now been found that recombinant human uteroglobin also binds to insoluble fibronectin.

When fibronectin converts to its insoluble form, it changes conformation and deposits on the surfaces of cells and on the extracellular matrix, where it may act as an anchor for polymorphonuclear leukocytes, macrophages, monocytes, and fibroblasts during an inflammatory episode. The receptors for fibronectin on cell surfaces are numerous and fall into different classes of molecules, including cell adhesion molecules (i.e. ICAM-1) and integrin complexes (i.e. alb4 integrin). These types of molecules mediate not only cell adhesion but also provide a means through which the cell senses, and reacts to, its environment. Recombinant human uteroglobin specifically binds to fragments of fibronectin that contain type III domains, and the RGD-containing type III domain (#10), in particular. The RGD peptide is well known as a mediator of cell adhesion in leukocyte extravasation during inflammation. RGD-containing peptides are potent inhibitors of cell adhesion in vitro, for many types of mammalian cells. Commercial interests have attempted to use RGD peptides and peptidomimetics as anti-inflammatory agents in vivo, but have met with limited success due to the instability of these types of compounds. Therefore, the potential use of recombinant human uteroglobin as an inhibitor of cell adhesion to fibronectin in vitro was investigated.

Cellular adhesion assays were performed essentially as described by Retta, et al. (“Adhesion to Matrix Proteins” in Methods in Molecular Biology, Dejana, E. and M. Corada, Eds., 96: 125-130, Humana Press, Totowa, N.J.; 1999). Briefly, a 96-well fibronectin-coated plate was blocked (to prevent non-specific binding of cells or proteins to the plastic) with 1× Pierce Blocker™, containing 5% BSA, at 37° C. in incubator for at least 1 hour. The blocking reagent was then removed and the wells were washed three times with PBS (phosphate buffered saline). The prepared plate were stored in the incubator while preparing the cells. Two cell lines, NIH 3T3 and Hela cells (ATCC # CCL-2) were selected for the assay. NIH 3T3 cells have been shown to have a high density of the uteroglobin receptor and Hela cells do not have the uteroglobin receptor. Both lines were grown with standard tissue culture techniques in Dulbecco's Modified Eagle's Medium containing 10% fetal calf serum. The cells were removed from culture flasks using 5 mM EDTA and spun down gently. The cells were then washed three times by alternately resuspending in serum-free medium and repeat centrifugation. The final cell pellet was resuspended to an estimated density of 2.5-7.5×10⁴ cells per 200 ml. Cells were partitioned into aliquots to receive either uteroglobin, a monoclonal antibody control, Type Ib secretory PLA2 (porcine pancreatic) (ppPLA₂), myoglobin (dog heart) control, or PBS control. Because uteroglobin and sPLA₂s share structural similarity, PLA₂ was tested as well.

Cells were then added to each well of the fibronectin-coated plate as quickly as possible. The plates were incubated at 37° C. in a CO₂ incubator for 1 hour. While the plated cells were incubating, live cells in the pipetted suspension were counted by trypan blue dye exclusion using a hemacytometer. At one hour the wells of the plate were aspirated and washed one time with PBS to remove non-adhered cells. The adhered cells were then fixed to the plate with 3.7% paraformaldehyde in PBS for 10 minutes at room temperature. The plates were then washed once with PBS and the adherent cells were stained by adding 200 ml of 0.25% R250 Coomassie blue to each well and allowing the cells to stand at room temperature for 1 hour. The stain solution was then aspirated and wells washed three times with PBS. Adherent cells were quantitated by reading the optical density at 540 nm in microtiter plate reader.

The tabulated results shown in Tables 8A & 8B below are reported as the percent inhibition of cellular adhesion. Percent inhibition was calculated as 100% minus the ratio of the mean OD of the test protein over the mean OD of the PBS alone (no protein) control. All protein groups were run in triplicate in each experiment. All numbers represent the mean percent inhibition for three separate experiments.

TABLE 8A
Effects of Uteroglobin on Cellular Adhesion to Fibronectin;
NIH-3T3 Cell line (high density of uteroglobin receptor(s);
mouse lung fibroblasts)

| | | Anti-fibronectin | | |
|---|---|---|---|---|
| | Uteroglobin | monoclonal | ppPLA₂ | Control |
| | (50 ng) | antibody (10 μg) | 50 ng) | (10 μg) |
| Percent Inhibition | 54% | 60% | 68% | 31% |

TABLE 8B
Effects of Uteroglobin on Cellular Adhesion to Fibronectin;
Hela Cell line (low density of uteroglobin receptor(s);
human cervical carcinoma)

| | | Anti-fibronectin | | |
|---|---|---|---|---|
| | Uteroglobin | monoclonal | ppPLA₂ | Control |
| | (50 ng) | antibody (10 μg) | (50 ng) | (50 ng) |
| Percent Inhibition | −5% | ND | 28.5% | 23% |

These results show that rhUG and ppPLA2 mediate a similar anti-adhesive effect, suggesting a common signalling pathway. Both proteins showed a significant effect at inhibiting adherence of NIH-3T3 cells to plated fibronectin and no significant effect on Hela cells. This indicates that rhUG may bind to the receptor that recognizes ppPLA2 in NIH-3T3 cells. Both the N-type (neural type) and the M-type (muscle type) PLA2 receptors are known to be expressed on NIH-3T3 cells, and since both rhUG and ppPLA2 share common structural patterns, it is possible that the UG receptor is a PLA2 receptor. This hypothesis is supported by the fact that the M-type PLA2 receptor is known to play a role in muscle contraction. Together with our discovery of the effects of rhUG on smooth muscle in the lungs of ventilated newborn piglets, these data strongly implicate the M-type PLA2 receptor in the rhUG signalling pathway. Thus, we believe that the M-type PLA2 receptor acts as a UG receptor in certain cellular and tissue-specific contexts. In addition, inhibition of cellular adhesion to fibronectin is a powerful pharmacologic effect that has been used as a screen for anti-inflammatory compounds that can inhibit the adherence of inflammatory cells such as neutrophils to the vasculature at sites of inflammation, as well as inhibit the extravasation of these cells into the inflamed tissues. Thus this is a powerful anti-inflammatory property of rhUG and would be expected to lead to the regulation and/or inhibition of neutrophil infiltration of damaged tissue.

### Example 4

### Effects of rhUG in a Perfused Rat Lung Model of Endotoxin-Induced Inflammation

Another study of the effects of rhUG on pulmonary inflammation was done in a perfused rat lung model of lipopolysaccharide (LPS)-induced inflammatory response developed by Uhlig, et al (1996) and Ljungman, et al. (1996). This model simulates one of the major pathways through which bacterial infection causes pulmonary dysfunction and is often used to explore the mechanisms of LPS-induced pulmonary inflammation, as well as to determine the ability of potential therapeutic agents to counter these mechanisms. In this model, lungs were excised from adult Norway rats and perfused with saline buffer for two hours. A total of 16 rats were treated in four groups of four animals each, as shown in Table 9.

TABLE 9

| | | |
|---|---|---|
| Group 1 | Vehicle control | N = 4 |
| Group 2 | LPS only | N = 4 |
| Group 3 | rhUG only | N = 4 |
| Group 4 | LPS + rhUG | N = 4 |

The lungs were initially ventilated and perfused for about 5 minutes with 50 milliliters of sterile physiologic saline to wash out remaining blood cells. Then the perfusate buffer was changed and bacterial endotoxin (LPS) and/or rhUG were administered via the perfusate. The concentration of LPS used was 5 mg/kg and the dose of rhUG was 20 mg/kg. After two hours of perfusion, the perfusates were collected and the perfusion was halted. Perfusates were stored frozen at −80° C. The airway resistance of the ventilated lungs was measured as described. The tracheal clamps were removed and broncho-alveolar lavage performed in order to recover extracellular lung fluids and proteins. The broncho-alveolar lavage (BAL) fluid was then stored at −80° C. BAL fluids were analyzed for rhUG content as well as the pro-inflammatory cytokine (TNF-alpha). Human UG concentration in BAL fluid was measured with the competitive UG ELISA as previously described. Rat TNF-alpha was measured by ELISA using a commercially available kit (R&D Systems, Inc.) Total protein in BAL fluids was measured using the micro-BCA protocol according to the manufacturer's instructions (Pierce Chemical Co.).

In this perfused lung system, the LPS and study compound can be given either by intratracheal instillation or via the circulatory system by addition to the perfusate. The LPS and the rhUG were administered via the perfusates in order to insure an even distribution of the agents in the lungs. When the LPS is administered via the circulatory route, the model is more representative of sepsis than it is of a local pulmonary infection. However, LPS is a potent pro-inflammatory compound and induces similar changes to the lungs and airways, regardless of the route of administration.

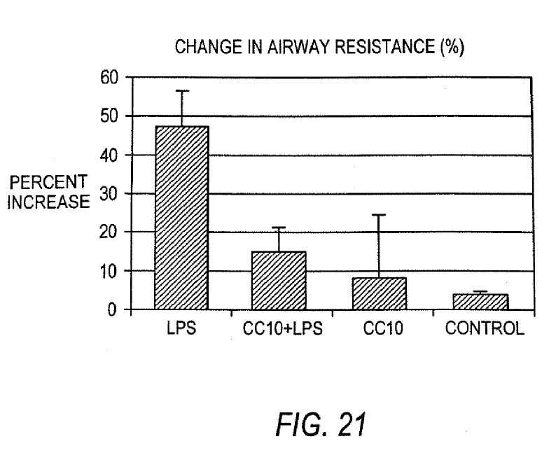

One of the changes that is common to the LPS response, irrespective of route of administration is an increase in airway resistance. The effect of LPS on airway resistance is shown in FIG. 21. The group receiving only LPS exhibits a large increase in airway resistance compared to the control. The group that received rhUG shows a modest increase in airway resistance that is probably due to the fact that the rhUG preparation itself, derived from a recombinant bacterial extract, contained a small quantity of endotoxin and other impurities. However, the group that received both the LPS and the rhUG shows only the same modest increase, demonstrating that the rhUG effectively reversed the effect of the LPS on airway resistance.

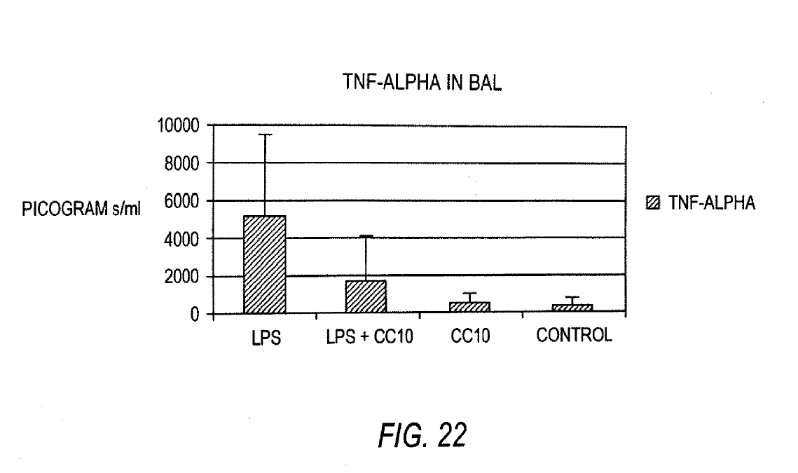

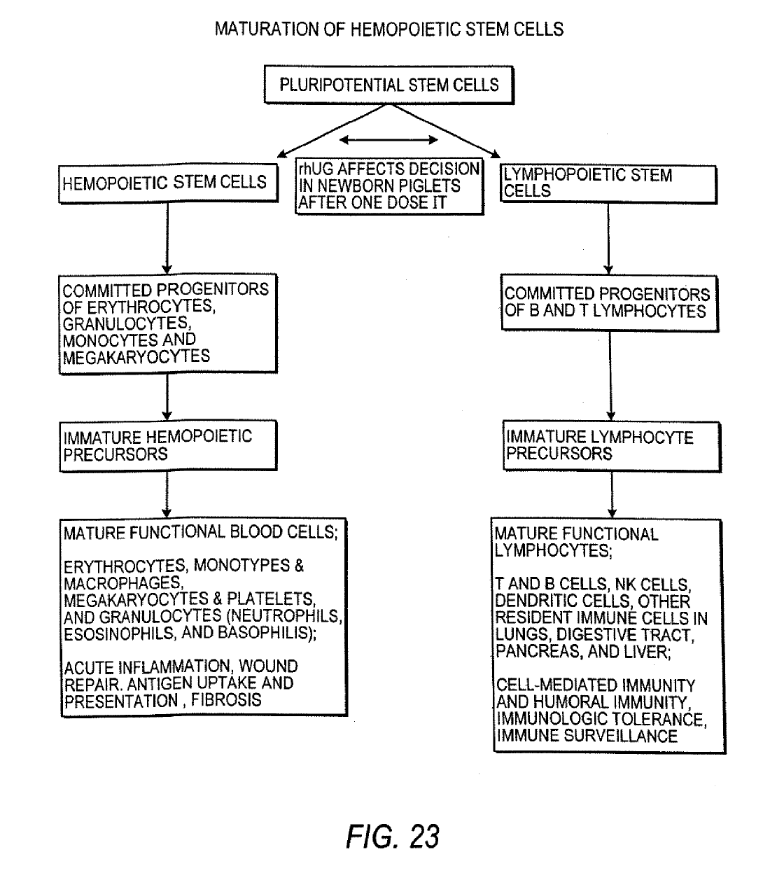

The effect of rhUG to mediate an inhibition of the release of the pro-inflammatory cytokine, TNF-alpha, by lung tissue into the extracellular lung fluid was also investigated. Elevations in TNF-alpha are a well-known sequelae to infection, and TNF-alpha is, in general, a marker of ongoing inflammation. The results are shown in FIG. 22. As expected, LPS mediated a large increase, over ten-fold, in the mean TNF-alpha concentration. The rhUG and control groups had comparable mean TNF-alpha concentrations, showing that rhUG mediated a significant decrease in the mean TNF-alpha concentrations in BAL fluid in LPS treated lungs.

These results on airway resistance are remarkable since very few, if any, agents have been shown to be so effective in countering this effect of LPS. The effect was achieved in the absence of any type of blood cell in the circulation and, therefore, is an effect of both agents directly on the lungs, airways, and vasculature. This is particularly unexpected for rhUG because the anti-inflammatory pulmonary effects of UG have previously been attributed to its inhibitory effects on white blood cells.

This result in perfused rat lungs is in agreement with the study in a newborn piglet model of oxygen toxicity where rhUG showed a similar effect on normalizing pulmonary compliance and distensibility (see Example 1). Without being bound by theory, it is believed that the beneficial effects of rhUG in this perfused rat lung model are not likely due to enhancement or protection of surfactant, as was also the conclusion in the ventilated newborn piglet model. These two experiments taken together also indicate that rhUG acts on the smooth muscle component of the vasculature and the airways to mediate relaxation, regardless of the particular type of inflammatory insult that triggers inappropriate or deleterious muscle contraction.

The concentration of rhUG in BAL fluid samples was determined. The mean rhUG concentration in each group is given in Table 10 below. Both of the groups receiving rhUG had substantial quantities of rhUG in the BAL fluids. The groups that did not receive rhUG had nothing but background. The limit of sensitivity for the assay is 5 nanograms/ml and anything less than that value is noise. Comparison of the values for CC10 concentration versus total protein concentration shows that these differences are not due to differences in the dilution factor inherent in the BAL fluids technique. The presence of rhUG in BAL demonstrates that UG is taken up from the blood by the airways and lungs.

Without being bound by theory, this suggests that a second source of UG in the body may replenish UG in the lungs when acute depletion of UG or Clara cells is caused by some external insult. It is possible to interpret the high circulating levels of UG in ALI and CLD as a physiologic response to compensate for decreased local pulmonary production of this essential protein.

TABLE 10

| | [CC10] | [Total protein] |
|---|---|---|
| Group | nanograms/ml | milligrams/ml |
| LPS | 1.3 +/− 0.3 | 6.2 +/− 8.5 |
| LPS + CC10 | 3,064 +/− 874 | 9 +/− 13 |
| CC10 | 2,590 +/− 362 | 1.6 +/− 0.4 |
| Control | 1.5 +/− 0.3 | 12 +/− 16 |

An additional treatment benefit shown in this study is that rhUG can be administered via the circulation and still mediate a therapeutic effect in the lungs, providing a practical route of administration to treat non-entubated patients with an acute or chronic lung condition. Thus, an intravenous injection could treat or prevent ARDS, even in sepsis patients with circulating LPS. An intravenous injection of rhUG could likewise treat bronchoconstriction in an asthma patient who is physically unable to inhale medication. Similarly, many patients with pulmonary fibrosis (IPF or CF) or chronic obstructive pulmonary disease (COPD) may have difficulty taking a breath, or inhaling other medication, relieved by an intravenous dose of rhUG. Finally, this study illustrates the potential for rhUG as an agent for the lowering of blood pressure caused by rigid, non-compliant, or non-elastic blood vessels, including primary pulmonary hypertension (PPH).

### Example 5

### Elevation of Neutrophils in UGKO Mice

In the course of determining baseline levels of complete blood counts and differential white blood cell counts in uteroglobin knock-out (UGKO) mice, a new phenotype of these mice was discovered. Whole blood was collected by retro-orbital bleed from six UGKO mice and six normal C57/Bl6 mice (the parental strain). The numbers of blood cells of different types were determined by automated cell counting, as well as by manual counting of cell types on differentially stained microscope slides. The data are shown in Table 11 below. Each number represents the mean of five determinations in five animals. In instances where determinations were not made in five animals, the numbers in parentheses indicate the number of animals for which determinations were made for that type of blood cell. There was a significant increase in the number of circulating neutrophils in the UGKO mice compared to the non-knockout parental strain. The UGKO mice had three times the normal level of neutrophils. This observation is significant in terms of determining the basis for the inflammatory necrotic foci in pancreas, spleen, and thymus, as well as the high level of circulating lysophosphatidic acid (LPA) and sPLA₂ activity (Zhang, 1997; U.S. Ser. No. 08/864,357) previously observed in these mice. A greater number of neutrophils could result in a greater tendency towards inflammation.

TABLE 11

| | | | Normal mouse |
|---|---|---|---|
| Cell Type | UG −/− (n = 5) | UG +/+ (n = 5) | Range |
| WBC | 13.46 K/μl | 5.8 K/μl | 4-12 K/μl |
| RBC | 10.6 M/μl (3) | 9.4 M/μl (4) | 7-11 M/μl |
| hematocrit | 51.3% (4) | 46.2% (4) | 35-45% |
| neutrophils | 19.2% | 9.5% (4) | 5-40% |
| lymphocytes | 74% | 75.6% | 30-90% |
| monocytes | 4% | 2.4% | 0-10% |
| eosinophils | 0.32% | 7.2% | 0-5% |
| basophils | 0% | 0% | 0-1% |

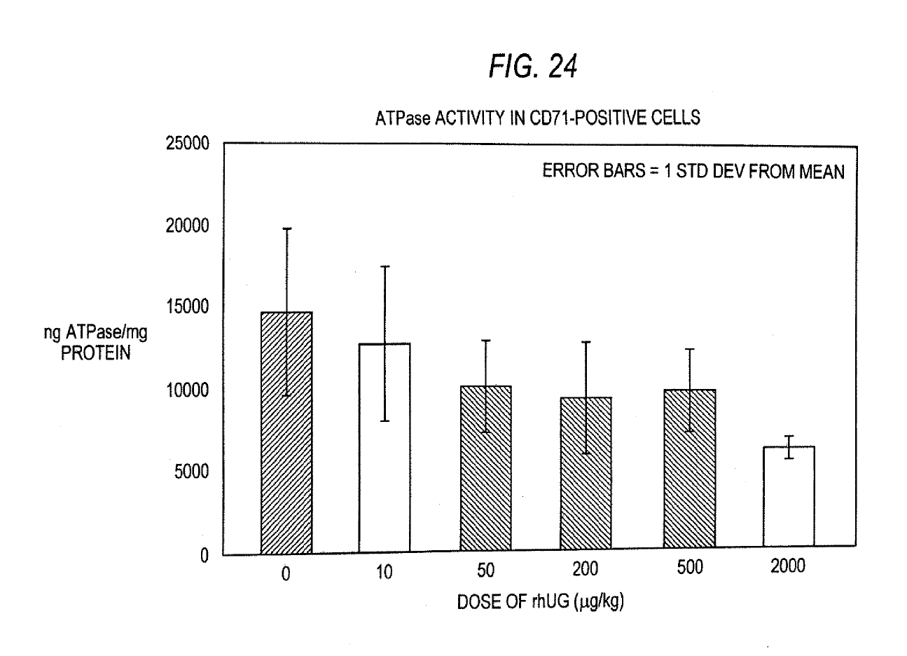

Taken together with the piglet data that demonstrated trends towards higher numbers of lymphocytes and lower numbers of PMN (quantitated one month after a single intratracheal dose of rhUG) this observation of high neutrophils in UGKO mice further shows that rhUG affects white blood cell distribution. In addition, there is another mouse model of IgA nephropathy, the DDY mouse, in which the renal fibrotic phenotype is due to abnormal B cell development. The necrotic foci in the spleen and thymus of the UGKO mice, organs where white blood cells normally differentiate into mature T and B cells, indicate that the absence of UG results in a defect in T or B cell maturation, not just granulocyte maturation, as shown in FIG. 24. Likewise, necrotic foci in the pancreas of the UG knockout mouse suggest a role for UG in maturation of resident lymphoid cells. Therefore, long term immunomodulatory effects of rhUG are due to alterations in distribution of white blood cells, both lymphoid and hemopoietic lineages, in the body. Long term anti-inflammatory effects of rhUG may be due to its regulation of production of hemopoietic precursors, including neutrophils (PMN). Long term effects of UG in preventing tumor formation are due at least in part to the increase in the number of lymphocytes that mediate immune surveillance.

These long term effects of UG are distinct from short term anti-inflammatory or immunomodulatory effects in that the long term effects involve immature precursors, rather than mature cells. Short term anti-inflammatory effects of rhUG are due to its immediate direct effects on mature pro-inflammatory leukocytes, such as inhibition of release of TNF-alpha and inhibition of chemotaxis. Furthermore, the nature of the effect is different than has been previously described because UG affects the maturation process itself, rather than suppressing a response to an external stimulus in the mature cells. These long term effects of UG indicate that the application of rhUG, and other UG supplementation strategies such as gene therapy and glucocorticoid administration, would be successful in the treatment of hemopoietic stem cell disorders.

### Example 6

### Short Term Effects of rhUG on Lymphoid and Hemopoietic Cell Metabolism In Vivo

Based on the piglet study and the phenotypes of the UG knockout (UGKO) mice, it was postulated that rhUG could induce a growth arrest in certain types of circulating lymphoid and hemopoietic cells in the body. The term hemopoietic refers to several cell types and their precursors as indicated in FIG. 24, specifically including granulocytes (neutrophils, basophils, eosinophils), erythrocytes, monocytes and macrophages, and megakaryocytes. The term lymphoid refers to T cells and B cells, specifically including T helper cells (CD4), T suppressor cells (CD8), NK (natural killer) cells, B cells, dendritic cells, and their precursors, as indicated in FIG. 24. The term lineage includes not only the mature, fully differentiated forms of these cells, but also their precursor cells that are not yet committed to a differentiation pathway.

In order to determine whether rhUG could mediate a growth arrest in circulating granulocytes and lymphocytes, the short-term effects of rhUG on white blood cell metabolism in vivo were investigated. rhUG was adminstered intravenously to 24 young adult male Wistar rats in six dose groups of four animals each, as shown in Table 12. The rhUG was given by tail vein injection in a constant volume of 200 microliters. Animals were housed in metabolism cages and given free access to food and water. The animals were anesthetized and sacrificed twelve hours after administration of a single dose of rhUG. During sacrifice, five milliliters of whole blood was collected into EDTA collection tubes. The blood from all animals in each group was pooled and kept at room temperature for 1-3 hours during processing.

TABLE 12

| Dose Group | Number |
|---|---|
| 0 μg/kg | 4 |
| 10 μg/kg | 4 |
| 50 μg/kg | 4 |
| 200 μg/kg | 4 |
| 500 μg/kg | 4 |
| 2,000 μg/kg | 4 |

Specific populations of white blood cells were purified from each pool of blood. Cells bearing surface epitopes for CD11b and CD71 were selected out and analyzed. The metabolic states of these cell types were then assayed by measuring total ATPase activity in extracts of these cells (see U.S. Pat. No. 5,773,232). Any change in the level of ATPase activity indicates that the cell is reacting to some signal or other change in its environment. In this experiment, conducted in the absence of an antigenic or mitogenic stimulus, the change in ATP level indicates the response of these cell types to exogenous intravenous rhUG only.

Pharmaceutical grade recombinant human UG, 5.5 mg/mL blood was collected in EDTA tubes (Becton Dickinson) from treated male Wistar rats. Tosyl-activated magnetic particles (Dynabeads M-450) were obtained from Dynal Biotech, and mouse monoclonal antibodies to rat CD11B, CD4, CD8, and CD71 cells were obtained from Research Diagnostics, Inc. Rabbit anti-mouse antibody (Pierce Chemical Co.) was used to coat the magnetic particles. All other reagents used are listed and described in the product inserts for each of the methods used in this study. Bead coating was done according to the manufacturer's instructions (Dynal Biotech Product Nos. 140.03 and 140.04, Dynal Inc.). Measurement of ATPase concentrations was also done according to the manufacturer's instructions (Cylex, Inc.) using the in vitro CMI™ Assay for T Cell Activation, Universal Test Kit, and CD4 in vitro CMI™ Assay for T Cell Activation. Protein determinations were done using the micro-BCA protocol from the BCA Protein Assay Kit according to the manufacturers instructions (Pierce Chemical Co.)

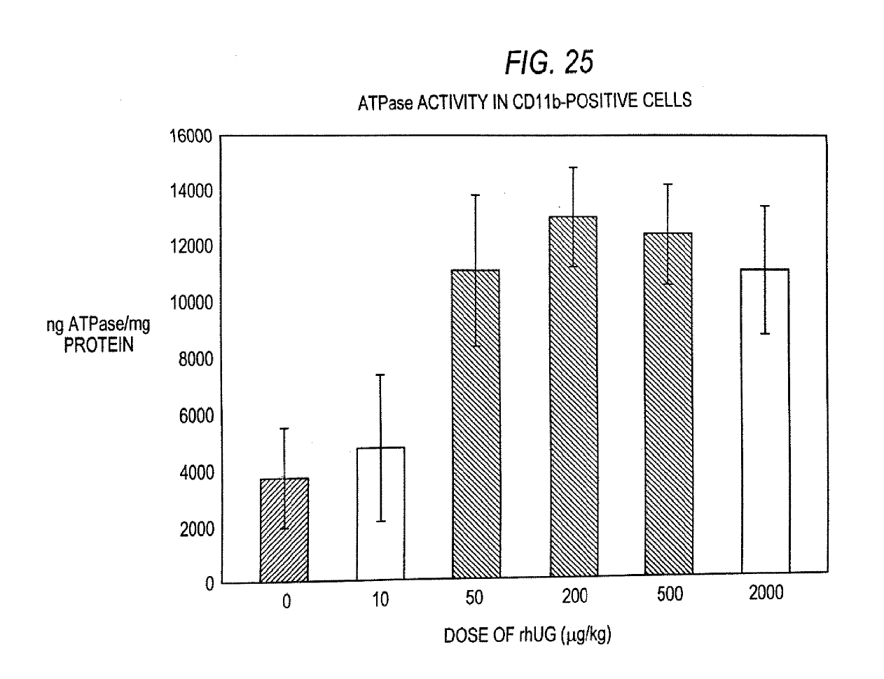

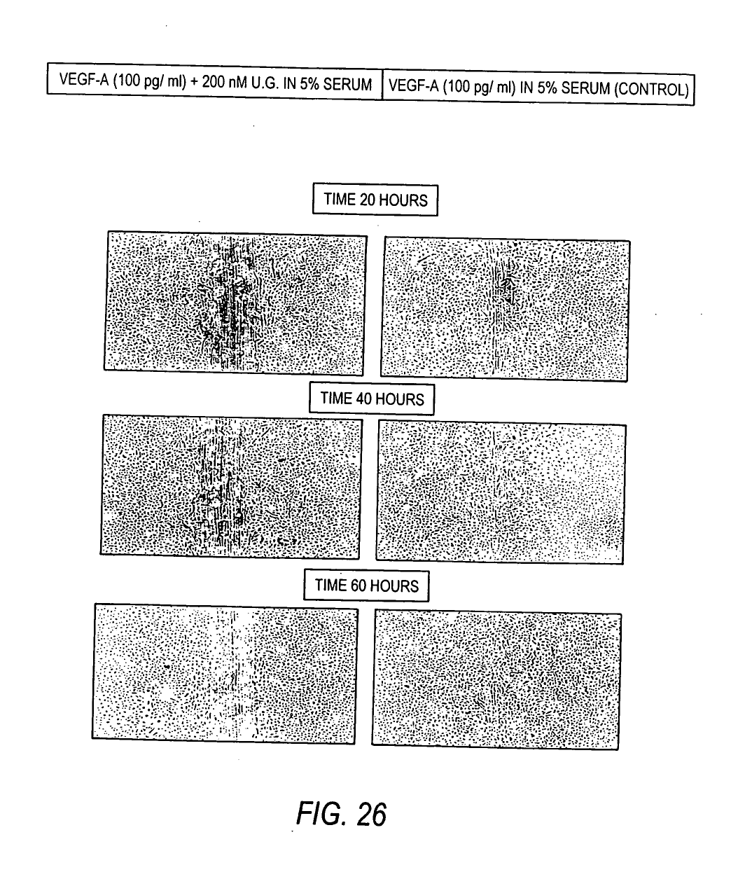

CD71 is a widely distributed cell surface marker for activated or proliferating cells and is the transferrin receptor required for iron uptake in all rapidly metabolizing cells. Proliferating lymphoid and myeloid cells in the circulation express CD71 and are specifically selected by the technique used. CD11b is another Cluster of Differentiation cell surface antigen also an integrin alpha chain, that is often present on the surface of neutrophils, monocytes and NK cells. CD11b is a marker for activated and adherent cells. It is upregulated during inflammation required for the firm adhesion of leukocytes to endothelial cell surfaces and subsequent extravasation from the blood into the tissues. Each set of cells was isolated from each pool of blood by aliquoting 100 μl to each of eight wells in a microtiter plate for each CD antigen (e.g., eight replicates of each magnetic bead separation). The cells pulled down by the antibody-conjugated magnetic beads were washed once with PBS and then an ATPase extract was prepared and analyzed. The results for each CD cell type for each of the six dose groups is shown in FIGS. 25-26.

The data in FIG. 25 indicates that rhUG inhibits ATPase activity in CD71-positive cells in a dose dependent manner. This dose dependent decrease may reflect either a slowdown in the metabolism or an arrest of the cell cycle, in circulating cells such as T and B cells, or PMN or their precursors. Alternatively, it may indicate a dose dependent decrease in the number of proliferating cells that enter the circulation over the twelve-hour study period such as neutrophils, monocytes and their precursors. Since the high concentrations of UG in blood used in this study are not physiologic, except in instances of acute or chronic lung injury, this metabolic arrest or anti-proliferative effect may be a natural mechanism for down-regulating an ongoing inflammatory and immune response, affecting both lymphocytes and myeloid cells.

It has been found that the opposite result is true of cells that express the CD11b marker. In general, CD11b-positive cells are mature lymphocytes and leukocytes, as opposed to the majority of the CD71-positive cells which may be immature. FIG. 26 shows that rhUG enhances ATPase activity in CD11b-positive cells in a dose dependent manner that saturates at and above 50 μg/kg. The nature of this stimulation is either an increase in metabolic activity of an unchanged number of circulating cells or an increase in the number of cells in the circulation. This result was surprising since mature neutrophils represent the majority of cells in the circulation that rapidly become CD11b-positive during an inflammatory response.

### Example 7

### Effects of rhUG on Human Vascular Endothelial Cells Stimulated with VEGF

### Introduction

Pathological angiogenesis plays a critical role in the clinical progression of solid tumors and participates in the pathogenesis of numerous inflammatory and ischemic disorders (Carmeliet and Jain, 2000). Considerable efforts are being made by academic and industrial investigators to identify novel pharmacological agents that can modulate angiogenesis in vivo (Carmeliet and Jain, 2000; Eatock et al., 2000; Hagedorn and Bikfalvi, 2000; Klohs and Hamby, 1999; Zhu and Witte, 1999).

Vascular endothelial growth factor (VEGF) and other related growth factors have been identified as critical for tumor growth and angiogenesis and as potential therapeutic targets (Schlaeppi and Wood, 1999; Gerwins et al., 2000; McMahon, 2000; Frelin et al., 2000; Veikkola et al., 2000; Ferrara and Alitalo, 1999b; Ferrara, 1999a; Arii et al., 1999; Crew, 1999).

Other important factors involved in regulating pathological angiogenesis are cellular integrins, especially alpha-v beta-1 and alpha-v beta-3 and extracellular matrix (ECM) components, especially fibronectin (Beckner, 1999; Chandrasekaran et al., 2000; Kim et al., 2000; Ruoslahti, 1999; Maier et al., 1999; Soldi et al., 1999; Collo and Pepper, 1999; Grant et al., 1998; Takei et al., 1998). Fibronectin binds alpha-v beta-1, which in turn modulates alpha-v beta-3 (Kim et al., 2000). Integrin activation by VEGF (Byzova et al., 2000) and modulation of VEGF receptors by integrins (Soldi et al., 1999) have been described. This suggests that complex autocrine loops involving ECM-integrin signaling and growth factor receptor signaling modulate angiogenesis.

This example reports the results of an investigation to determine whether rhUG had anti-angiogenic properties in standard in vitro models of angiogenesis (VEGF-induced migration and “lesion repair” assays). It has been found that:
1) rhUG inhibits VEGF-induced migration of primary human microvascular endothelial cells (HMEC) at nanomolar concentrations
2) rhUG inhibits VEGF-induced migration of HMEC only in the presence of fibronectin
3) rhUG inhibits invasion of soft agar by A549 cells, a transformed cell line derived from a human non-small cell lung carcinoma (NSCLC)

Taken together, these observations indicate that rhUG inhibits human primary endothelial cell migration, a key determinant of angiogenesis, through a novel mechanism that requires the presence of fibronectin, and most likely the formation of a fibronectin/UG/receptor complexes. These findings suggest that rhUG is a candidate new drug for the treatment of pathological angiogenesis in cancer and other disorders.

### Materials & Methods

### Cell Culture.

Human foreskin dermal microvascular endothelial cells (HMVEC) were obtained from Cell System Corp. (Kirkland, Wash.), grown in EGM-2 medium (Clonetics, San Diego, Calif.), and used from passages 2 to 10. A549 and HEC-1A cell lines were obtained purchase from ATCC and cultured in F12 HAM medium with 10% fetal bovine serum (FBS) and McCoy's modified medium with 10% FBS, respectively (GIBCO BRL., Grand Island, N.Y.).

### Cytotoxicity Assay.

Human foreskin dermal MVEC, A549 and HEC-1A were used to study a possible cytotoxic effect of human recombinant uteroglobin (rhUG). Each cell type was cultured in media containing 10% FBS. Cells were harvested by trypsinization and then plated in a 96-well plate at 30,000 cells per well in 200 μl. After overnight incubation, cells were treated with 1 μM rhUG. At different time points (22 to 90 hours) cells were washed with 1×PBS and fixed with 50% cold TCA (trichloracetic acid. After 1-hour incubation, wells were washed (4-5 times with ddH₂O), air-dried, and rinsed with 100 μl/well sulfo-rhodamine B stain (SRB; 04% in 1% acetic acid) for 30 minutes. After removal of SRB, wells were washed (4-5 times with 1% acetic acid), dried and 200 μl of Tris base was added in each well. The plates were shaken for 5 min. Finally, the 96-well plate was read at 490 and 530 nm using an ELISA reader. Each point was run in quadruplicate per each cell type.

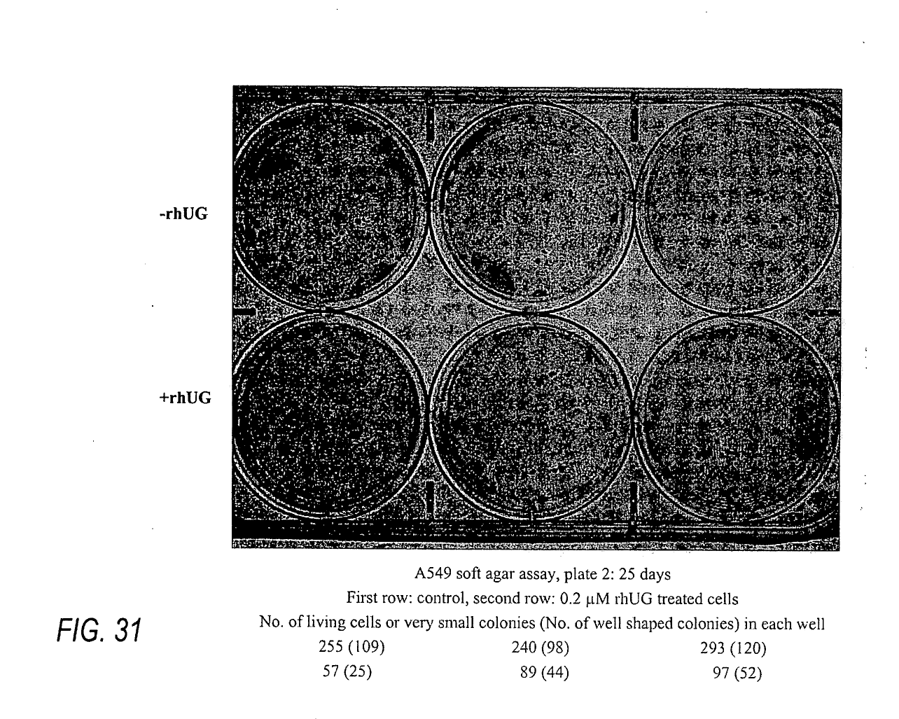

The rhUG had no effect on the viability of MVEC cells. This is shown in FIG. 31 where HMVEC, A549 and Hec-1A cells grew as well with 1 μM rhUG in the medium as without any rhUG. These results prove that rhUG does not have any cytotoxic effects, nor does it inhibit proliferation of these cells in the absence of a growth factor (VEGF) or other mitogen. This result implies that successful treatment of cancer in vivo with rhUG requires an additional apoptotic signal or cytotoxic agent such as a chemotherapeutic agent.

### Endothelial Cell VEGF Stimulated Proliferation Assay.

To study a possible inhibitory effect of rhUG as an inhibitor of endothelial cell proliferation, human foreskin dermal MVEC (Cell System Corp., Kirkland, Wash.) were used. Cells were harvested by trypsinization, resuspended in EGM-2 (Clonetics, San Diego, Calif.), and plated in a 6-well plate at 80,000 cells per well in 200 μl. After overnight incubation, the media were replaced with fresh EBM-2 with 1.5% FBS. After a 4 hour starvation period cells were treated with recombinant human VEGF-A (R & D Systems, Minneapolis, Minn.) plus a variable amount (0.05 to 1 μM) of rhU.G. The final concentration of recombinant human VEGF-A was 10 ng/ml. At 48 and 96 hours the cells were quantitated using a hematocytometer.

An alternative approach was used for the same purpose. Human foreskin dermal MVEC were plated in a 96-well plate at a density of 10,000 cells per well. After the treatment described above, the cells were washed with 1×PBS and rinsed with 50% cold TCA. After I-hour incubation wells were washed (4-5 times with ddH2O), air-dried and rinsed with 100 μl/well SRB (0.4% in 1% acetic acid) for 30 min. After removal of SRB, wells have been washed (4-5 times with 1% acetic acid), air-dried, treated with 200 μl/well Tris base, and were placed on a gyratory shaker for 5 minutes for gentle mixing. Finally, the 96-well plate was read at 490 and 530 nm using an ELISA reader. Each point was run in a set of eight replicates.

The results of the both approaches are consistent with each other. The results of a representative experiment are shown in FIG. 31b. In contrast to the lack of anti-proliferative activity in the absence of VEGF-A, rhUG counteracted the stimulation of proliferation that was induced by VEGF. These data, together with the cytoxicity assay, demonstrate that rhUG does not inhibit normal growth of non-transformed cells, but does abrogate the effects of mitogen or growth factor-stimulated proliferation. The potent inhibitory effects of rhUG on accelerated or abnormal proliferation without simultaneous inhibition of normal, unenhanced proliferation. This implies that rhUG could impart the benefits of chemotherapeutic agents without the toxic effects. In vitro wound healing assay (proliferation-migration assay).

Human foreskin dermal MVEC were grown to confluence in 10 cm cell culture dishes. Post-confluent HMVEC were wounded by gently scraping the floor of the dish with a serological pipet. Three uniform injuries were produced by a single pipet scrape across the cell monolayer (as examined by optical microscopy) in each dish. After washing with 1×PBS, fresh media were added and two different medium conditions were tested (1.5 and 5% FBS). After 4.5 hours, post-confluent cells were exposed to 0.2 μM rhUG. After 30 min., cells were exposed to 100 pg/ml VEGF-A (R & D Systems, Minneapolis, Minn.). At regular intervals between post-injury hour 22 to 110, cells were examined by optical microscopy. Under each condition, each time point was performed in quadruplicate and the control represented by cells not exposed to rhUG.

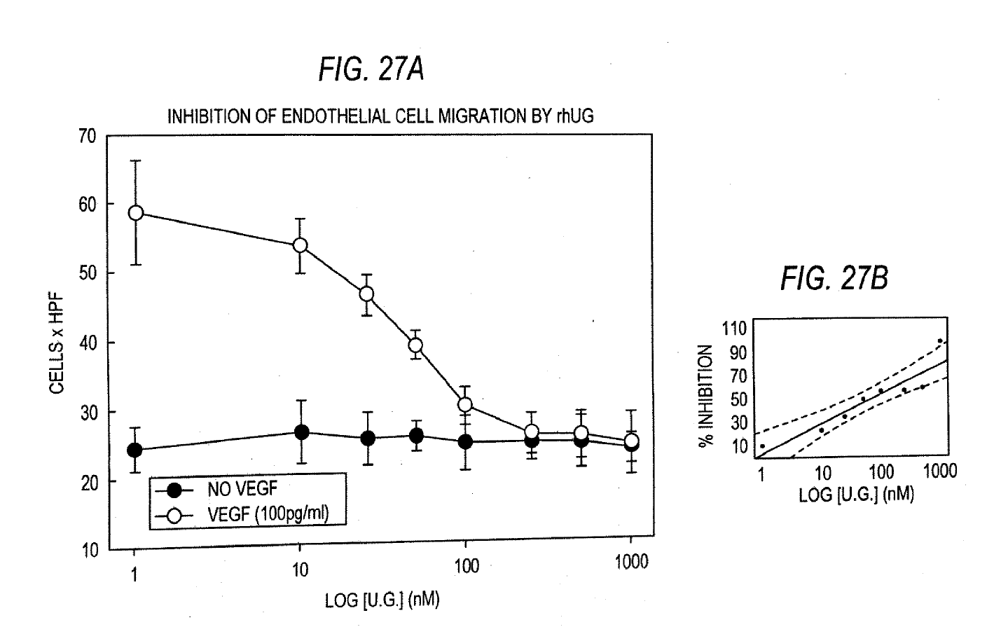

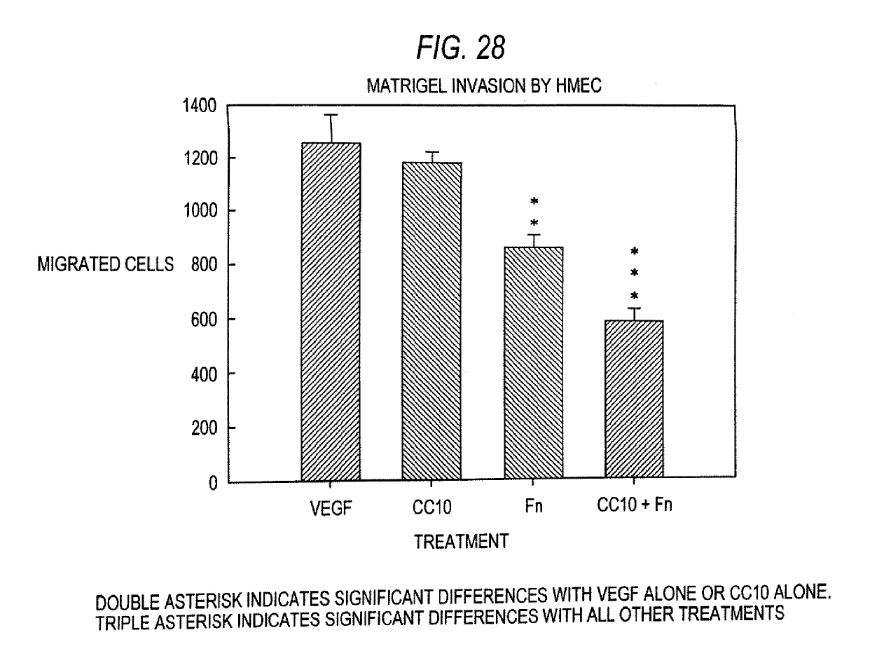

In the absence of rhUG, the MVEC migrate into the injured area that was cleared of cells by the pipet scraping technique. In the presence of 200 nanomolar rhUG, the migration of VEGF-stimulated MVEC was significantly inhibited. Photographs of representative injury areas showing MVEC migration were taken at 110 hours post-injury and these results are shown in FIGS. 27 and 28.

### Endothelial Cell Migration Assay.

Human foreskin dermal MVEC (Cell System Corp., Kirkland, Wash.) were cultured in EGM-2 medium (Clonetics, San Diego, Calif.) up to 80% of confluence. The medium was replaced and the cells starved overnight in EBM-2 containing 0.1% FBS. The day after, the endothelial cells were harvested, resuspended into DMEM (Dulbecco's Modified Eagle's Medium) with 0.1% FBS, plated on the bottom side of a modified Boyden chamber (Nucleopore Corporation, MD), and allowed to attach in the inverted chamber for 1.5 hours at 37° C. The chamber was then re-inverted and rhUG added at different concentrations in the presence of VEGF-A (R & D system, Minneapolis, Minn.) to the wells of the upper chamber. The cells were allowed to migrate for four hours at 37° C. Membranes were recovered, fixed and stained, and the number of cells that had migrated to the upper chamber counted in ten high power fields. Background migration to DMEM plus 0.1% FBS was subtracted and the data reported as the number of cells migrated per 10 high power fields (400×) or, when results from multiple experiments were combined, as the percentage of maximal migration to a positive control. Each concentration of rhUG was tested in quadruplicate. VEGF-A was tested at 100 pg/ml and neutralizing antibodies to VEGF-A were used as control at 20 μg/ml.

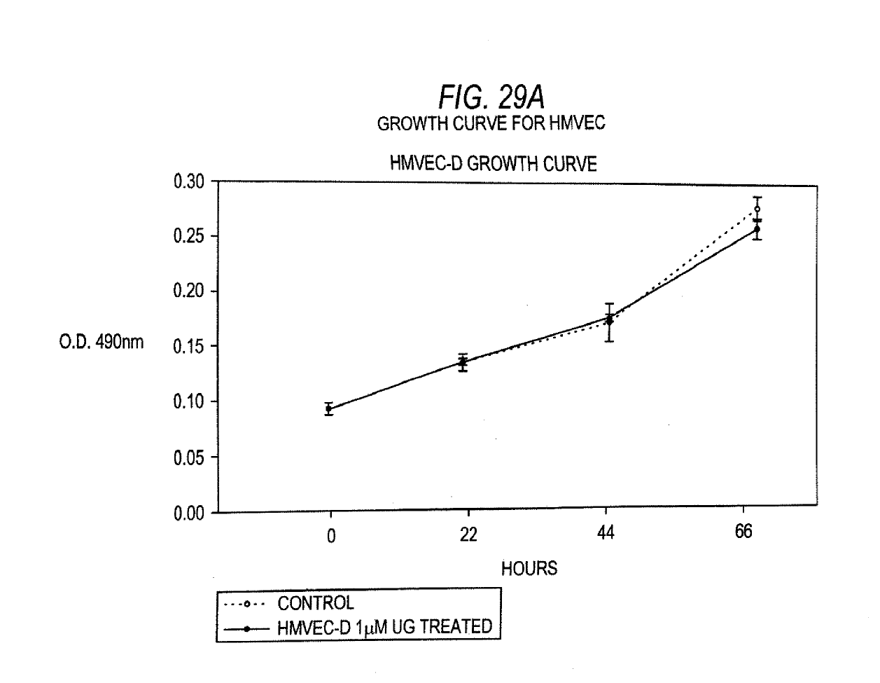

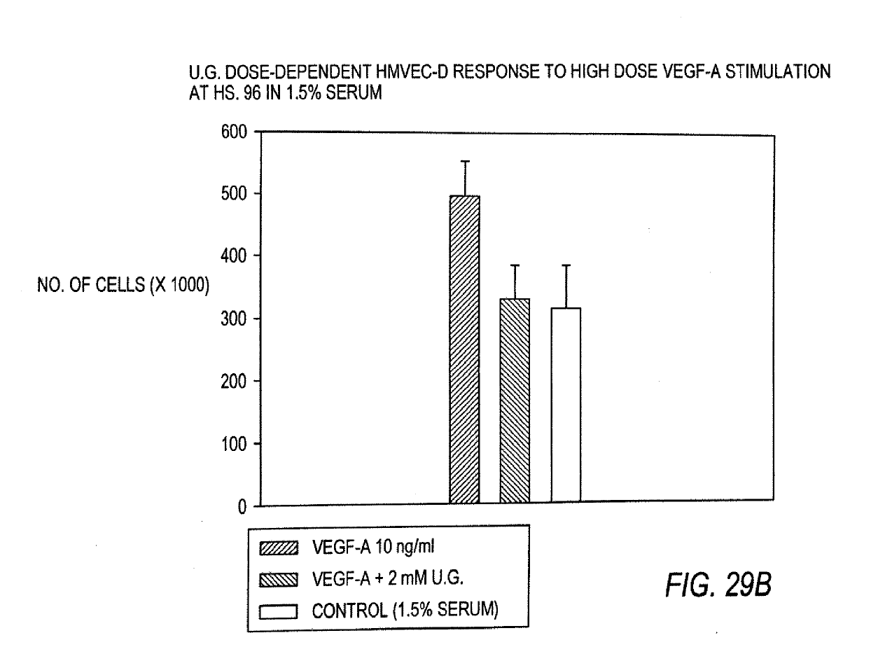

The effect of rhUG on the VEGF-A-stimulated migration of human dermal MVEC is shown in FIG. 29. This is a standard assay that is used to screen agents that inhibit angiogenesis, since one of the first steps in new blood vessel development is the migration of endothelial into a new area. Our data clearly show that rhUG mediates a profound effect in inhibiting the MVEC migration at nanomolar concentrations of rhUG. This result is even more important because we've shown in our animal studies that these rhUG concentrations are non-toxic, therapeutically, pharmacokinetically approachable, deliverable by several routes of administration, and physiologically relevant in that they can mediate significant biological effects.

### Western Blot for Fibronectin Detection.

Western blot analysis was performed on Hfl-1 cells, A549 and Hec-1A and fibronectin cross-reactive species were detected with an anti-human fibronectin monoclonal antibody in order to determine the endogenous expression of fibronectin. Whole cells were cultured (as described in Example 8), scraped from the flask and transferred to an Eppendorf tube. An equal volume of SDS-PAGE buffer (Novex) was added to each sample and mixed by vortexing. The samples were then heated for 10 minutes at 85° C. and loaded onto a 10% Tris-glycine SDS-PAGE gel (Novex). Rainbow Marker™ (Amersham) was run as the size standard. Blocking, probing, and washing were done as described in Example 8, except that a monoclonal anti-human fibronectin antibody was used as the primary antibody at a dilution of 1:2000 and a rabbit anti-mouse IgG-HRP conjugate was used as the secondary antibody at a dilution of 1:5,000.

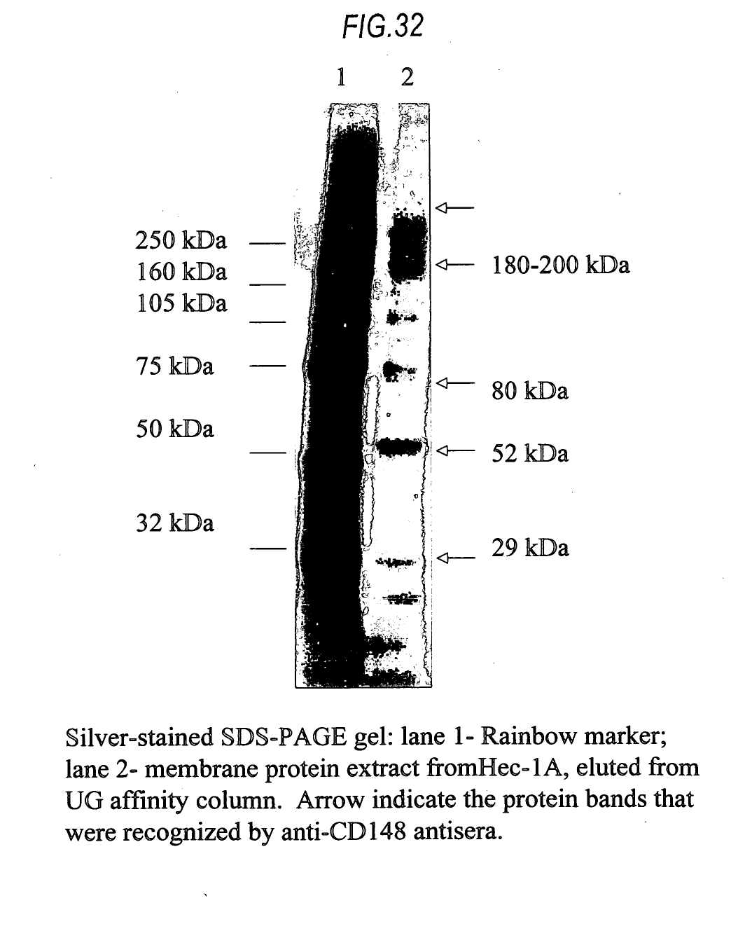

Results are shown in FIG. 32. All cells line made proteins that were recognized by anti-human fibronectin antibody. Hfl-1, the non-transformed cell line, exhibited a very high molecular weight complex that may be 440 kDa which corresponds to the size of an intact fibronectin dimer. Both of the transformed cell lines make immunoreactive protein, the highest band of which is about 220 kDa, corresponding to a fibronectin monomer. There are several other immunoreactive bands that probably represent proteolytic cleavage products of the fibronectin monomer. Thus all three of these cell lines synthesize fibronectin, and fragments derived from it, or are proteins that are recognized by anti-fibronectin mAb. These cell lines therefore, synthesize fibronectin and fibronectin-like proteins that make them capable of responding to rhUG without the addition of exogenous fibronectin.

### Soft Agar Assay.

Recombinant human UG-treated A549 cells were analyzed in a soft agar assay. Two 6-well plates were poured with 3 ml of 0.6% agar 1×F12 HAM medium (GIBCO BRL, Grand Island, N.Y.) containing 10% FBS. A concentration of 0.2 μM of rhUG was used in each well. The plates were allowed to equilibrate overnight at 37° C., then 12 glass tubes were filled with 0.3% agar F12 HAM medium with 10% FBS. 45,000 cells per sample were added to each tube at 38° C. The cells plus agar in the tubes were then poured onto the agar on the plates and the plates were incubated in a cell incubator under standard conditions (37° C., 5% CO₂) for 35 days. Every 5 days, a fresh aliquot of rhUG was added to a concentration of 11M in a fixed volume of 150 μl of fresh medium to each of the 6 test wells after expired media was removed by aspiration. The 6 control wells received the same amount of fresh medium lacking rhUG at the same times. At the end of the experiment, a 300 μl aliquot of rhUG was added to the surface of each culture to a concentration of 1 μM and the cells were incubated at 37° C. for an additional 36 hours. The excess liquid was then aspirated off and the colony staining characteristics were examined by optical microscopy. Colonies were stained with SRB dye as described above.

Each plate was examined, photographed, and colonies were counted. The results are shown in FIG. 33 for A549. The rhUG inhibited the invasion of the soft agar by A549 by over two-fold. This demonstrates the ability of rhUG to reduce extracellular matrix invasion by A549, and cancer cells in general.

Matrigel Invasion Assay with Fibronectin

HMEC were grown in complete medium as described in the above wound healing assay. The assay was performed in serum-free medium. Cells (5000 per well) were incubated for 24 hours in Boyden chambers and tested for invasion through Matrigel-coated filters (Nucleopore). The bottom chamber medium contained VEGF (100 pg/ml) in all wells. The top chamber medium contained CC10 at the IC₅₀ for migration inhibition (100 nM), plasma fibronectin (Sigma) at the same concentration (100 nM) or a combination of Fn and CC10. After 24 hours, attached but non-migrated cells were wiped off the upper face of each filter with Q-tips and migrated cells attached to the bottom face of each filter were stained and counted by two blinded observers. Data represent averages of four intermediate power fields (magnification ×100) counts plus or minus standard deviations. Data were analyzed by one-way ANOVA with Tukey correction for multiple comparisons

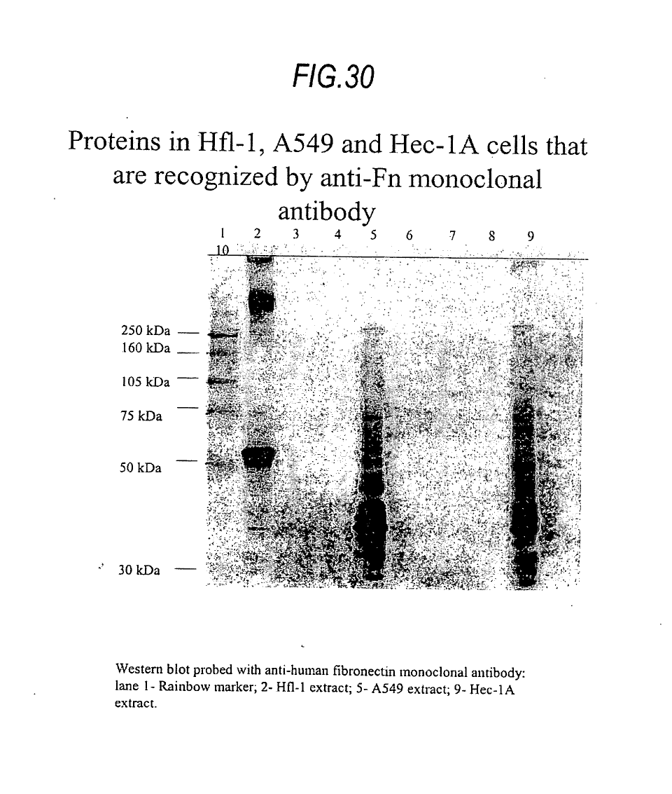

As shown in FIG. 30, CC10 alone had no significant effect on Matrigel invasion in serum-free medium. Fn alone had a modest but statistically significant inhibitory effect. However, CC10 plus Fn inhibited invasion to a significantly higher extent than Fn alone or CC10 alone (p<0.05). Thus, CC10 requires the presence of Fn to inhibit Matrigel invasion by human primary microvascular endothelial cells. This is consistent with the observation that no saturable cell binding by CC10 was observed in the absence of Fn. These data indicate that CC10 binds HMEC as a complex with Fn and modulates Fn signaling. Qualitatively, the morphology of these cells incubated with Fn and CC10 together appeared in groups with intercellular adhesion. Cells incubated with Fn alone showed much less pronounced intercellular adhesion. Cells incubated in control medium or CC10 alone appeared mostly as individual cells. This suggests that upon binding Fn/CC10, cells modulate adhesion molecules and increase intercellular contacts, which may participate in the inhibitory effects observed.

The property of intercellular adhesion to cells of the same lineage is a property of non-transformed normal cells and is referred to as isotypic adhesion. Agents that promote isotypic adhesion are candidates for clinical drug development as anti-cancer agents. Isotypic adhesion of A549 cells transfected with a human uteroglobin expression vector has been reported (Szabo, 1996), but the involvement of fibronectin was not previously identified. The effect in this cell line was attributed solely to the expression of human UG, since the effect was not observed in the mock-transfected controls. However, we have observed that non-transfected A549 and Hec-1A cells grown under similar culture conditions also express proteins and protein fragments that are recognized by an anti-human fibronectin monoclonal antibody, as shown in FIG. 32. Indeed, many cell lines produce fibronectin immunoreactive proteins and protein fragments (Ruoslahti, 1987).

### Example 8

### Identification of UG Receptors

In a continuation of the effort to purify and identify the human UG receptor(s), originally described in PCT/US98/11026 two approaches were taken. The first was a bioinformatics approach in which the M-type PLA2 receptor and CD148 were identified as candidates for the UG receptor. The second was a continuation of the affinity purification approach. Human fetal lung fibroblast cell line, Hfl-1, was selected for two reasons: 1) It is a non-transformed diploid senescent cell line, and therefore, closer to a normal human cell than any of the tumor cell lines previously characterized as expressing UG binding proteins, and 2) PDGF-induced migration of Hfl-1 cells was reported by Lesur et al. (1995), suggesting the presence of a functional UG receptor.

Hfl-1 was obtained from the American Type Culture Collection, Inc. and cultured according to the supplier's instructions. Cells were cultured to approximately 90% confluence, then the media was decanted and the cells washed twice with sterile saline. Cells were scraped from culture flasks and lysed in 10 mM Tris buffer pH 7.5 with 1% Triton X-100 and Complete anti-protease (Roche). Unlysed cells were removed by centrifugation and the supernatant was loaded onto a UG affinity column. The UG affinity column was prepared by coupling 50 milligrams of substantially pure rhUG to 10 mls of NHS-Sepharose, according to the manufacturer's instructions (Pharmacia). The column was equilibrated either with 10 mM Tris, pH 7.5, 0.1% Triton X-100. After loading the crude cell lysate onto the column, the unbound proteins passed through the column by washing with cell lysis buffer. Bound proteins eluted in batch using 0.5 M NaCl in 10 mM Tris, pH 7.5 with 0.1% Triton X-100. Several proteins or protein fragments were retained by the UG affinity column. Proteins were visualized by Silver stain of a 10% Tris-Glycine SDS-PAGE gel (Invitrogen Corp.), as shown in FIG. 34.

In order to verify the presence of the M-type PLA2 receptor and CD148 in various UG-responsive cells, including Hfl-1, we generated rabbit polyclonal antisera against peptides derived from them. The peptides were synthesized and rabbit antisera raised using standard methods (Research Genetics, Inc.). The peptide derived from the M-type PLA2 receptor to which antisera was raised is: QNWDTGRERTVNNQSQR (SEQ ID NO. 2). The peptide derived from the CD148 protein to which antisera was raised is NGTDGASQKTPSSTGPSPVFD (SEQ ID NO. 3). Both of these peptides produced high titre antisera within three months.

The anti-M-type PLA2 receptor antisera and the anti-CD148 antisera were then used to analyze the crude extract and UG affinity-purified protein by Western blot. Equal volumes of crude lysates of Hfl-1 and UG affinity-purified proteins were run on 10% SDS-PAGE Tris-glycine gels (Invitrogen Corp.) with a Rainbow™ marker (Amersham Pharmacia, Corp.) using the manufacturers procedures. Gels were blotted to Hybond-P™ (Amersham Pharmacia, Corp.) using the Novex Xcell™ II apparatus according to instructions (Novex). The blots were blocked in 5% BSA (Sigma Co.) overnight at 4° C. Excess blocking solution was washed off with two washes in PBS with gentle shaking at room temperature. All following steps were performed at room temperature. Primary incubations with the rabbit antisera against the human M-type PLA₂ receptor and CD148 were done on separate blots containing identical protein samples. The blots were incubated in parallel with primary antisera dilutions of 1/500 in 5% BSA, 1×PBS, 0.2% Tween 20 for 1.5 hours at room temperature with gentle shaking. The primary antisera was then washed off with three washes in PBS, 0.1% Tween 20 with gentle shaking for 5-10 minutes each. Secondary incubations were goat anti-rabbit IgG conjugated to horse radish peroxidase (Pierce Chemical Co.) at 1/5000 in 5% BSA, 1×PBS, 0.1% Tween 20 for 1.5 hours at room temperature with gentle shaking. Three washes were then done in 1×PBS, 0.1% Tween 20 for 5-10 minutes each. The blots were developed using the ECL™ kit (Amersham Pharmacia Corp.) and viewed by autoradiography using Kodak Biomax™ ML film.

Protein bands corresponding to bands detected on each Western blot are indicated by arrows in FIG. 34. Similar protein staining and Western band patterns were obtained for three additional cell lines, also obtained from ATCC and cultured under the recommended conditions. These included: HL-60, HEC-1A, and A549. These UG binding proteins include the bands previously identified as UG receptor binding protein (Kundu, 1998), and some new protein bands have appeared. Proteins that eluted from the UG affinity column include both the M-type PLA2 receptor and CD148, as well as fragments derived from each, or otherwise antigenically related proteins.

The above description of the invention is intended to be illustrative and not limiting. Various changes or modifications in the embodiments described may occur to those skilled in the art. These can be made without departing from the spirit or scope of the invention.

### REFERENCES

1. Levin, S. W. et al., Life Sci. 38: 1813-1819 (1986);
2. Singh G. et al., Biochem. Biophys. Acta. 1039: 348-355 (1990);
3. Mantile, G. et al., J. Biol. Chem 268: 20343-20351 (1993);
4. Singh, G. et al., J. Histochem. Cytochem. 36: 73-80 (1987);
5. Bernard, A. et al., Clin. Chem. 38: 434-435 (1992);
6. Dhanireddy, R. et al., Pediatric Res. 23: 463A (1988);
7. Dhanireddy, R. et al., Pediatric Res. 33: 323A (1993);
8. Piomelli, D., Op. In Cell Biol. 5: 274-280 (1993);
9. Krishnan, R. S. et al., Science 158: 490-492 (1967);
10. Beier, H. Verhandl Deut. Zool. Ges. Heidelberg (1968);
11. Umland, T. C. et al., Nature Stuct. Biol. 1: 538-545 (1994);
12. Hard, T. et al., Nature. Struct. Biol. 2: 938-989 (1995);
13. Umland, T. C. et al., Nature Struct. Biol. 2: 919-922 (1995);
14. Stripp, B. R. et al., Am. T. Physio. 271 (Lung Cell. Mol. Physiol. 15): L656-L664 (1996);
15. Lesur, O. et al., Am. T. Respir. Crit. Care Med. 152: 290-297 (1995);
16. Glaser, K. B., Adv. Pharmacol. 32: 31-66 (1995);
17. Tykka, H. T. et al., Scand. J. Gastroenterol. 20: 5-12 (1985);
18. Sheuer, W., Klin. Wochenschr. 67: 153-159 (1989);
19. Barnes, H. J. et al., J. Mol. Biol., Feb. 23, 1996;
20. Aoki, A. et al., Mol. Hum. Reprod. 2: 419-497 (1996);
21. Anderson and Kurkland, Microbiological Reviews 54: 198-210 (1990);
22. Miele, L. et al., J. Biol. Chem. 265: 6427-6435 (1990);
23. Coalson, J. J. et al., Exp. Mol. Pathol. 37: 355-360 (1982);
24. Nagy, A. et al., Proc. Natl. Acad. Sci. 90: 8424 (1993);
25. Capecchi, M. R., Science, 244: 1288 (1989);
26. Harlow, E. and Lane D. Antibodies: A Laboratory Manuel, 1st Ed. Cold Spring Harbor Laboratory Press, Cold Spring Harbor, N.Y., 1988;
27. Mantile, G. et al., J. Biol. Chem. 267: 20343 (1993);
28. Ruoslahti, E. Ann Rev. Biochem. 57: 375 (1988);
29. R. O. Hynes, Fibronectins, New York: Springer-Verlag (1990);
30. Chemousor, M. A. et al., J. Biol. Chem. 266: 10857 (1991);
31. Zhang, Q. et al., J. Cell. Biol. 127: 1447 (1994);
32. Wu, C. et al. Cell 83: 715 (1995);
33. Zhang, Q. et al., J. Biol. Chem. 271: 33284 (1996);
34. Border, W. A. et al., J. Clin. Invest. 90:1 (1992);
35. Peri, A., et al., J. Clin. Invest. 92: 2099 (1993);
36. Peri, A. et al., J. Clin. Invest. 96: 343 (1995);
37. Oh, E. et al., Proc. Natl. Acad. Sci. (USA) 78: 3218 (1981);
38. Mosher, D. F. et al., Curr, Biol. 4: 810 (1992).
39. R. S. Krishnan, J. C. Daniel Jr., Science 158, 490 (1967).
40. H. M. Beier, Biochim Biophys Acta 160, 28 (1968).
41. A. Peri, E. Cordell-Miele, L. Miele, A. B. Mukherjee, J. Clin Invest 92, 2099 (1993).
42. G. Singh et al. Biochim. Biophys. Acta 950, 329 (1988).
43. J. Jackson, R. Turner, J. N. Keen, R. A. Brooksbank and E. H. Cooper, J. Chromatogr. 452, 359 (1989).
44. M. J. Beato, Steroid Biochem. 7,327 (1976); M. Gillener et al., J. Steroid Biochem. 31, 27 (1988).
45. K. Diaz Gonzalez and A. Nieto, FEBS Lett. 361, 255 (1995).
46. M. A. Watson and T. P. Fleming, Cancer Res. 56,860 (1996)
47. M. A. Watson, C. Darrow, D. B. Zimonjic, N. C. Popescu, T. P. Fleming, Oncogene 16 (2), 817 (1998).
48. L. Miele, E. Cordella-Miele, A. B. Mukherjee Endocrine Reviews, 8, 474 (1987).
49. L. Miele, E. Cordella-Miele, G. Mantile, A. Peri, A. B. Mukherjee J. Endocrinol. Invest., 17,679 (1994).
50. L. Miele, E. Cordella-Miele, A. Facchiano, A. B. Mukherjee, Nature 335, 726 (1988).
51. L. Miele, E. Cordella-Miele, J Biol Chem 265,6427 (1990).
52. G. Camussi, C. Tetta, F. Bussolino, C. Baglioni, J. Exp. Med. 171,913 (1990).
53. S. Lloret, J. J. Moreno, Biochem. Pharm. 50 (3), 347 (1995).
54. G. Mantile, L. Miele, E. Cordella-Miele, G. Singh, S. L. Katyal, A. B. Mukherjee, J Biol Chem 268, 20343 (1993);
55. G. Vasanthakumar, R. Manjunath, A. B. Mukherjee, H. Warabi, E. Schiffman, Biochem, Pharmacol. 37(3), 389 (1988).
56. R. Manjunath, R. et al. Biochem. Pharmacol. 36 (5), 741 (1987).
57. J. G. Vostal, A. B. Mukherjee, L. Miele, N. R. Shulman, Biochem. Biophys. Res. Commun. 165(1), 27 (1989).
58. A. Melchiori et al. Anticancer Res. 10(1), 37 (1990).
59. G. C. Kundu, G. Mantile, E. Cordella-Miele, A. B. Mukherjee, Proc. Natl. Acad. Sci. USA. 93, 2915 (1996).
60. K. Diaz Gonzalez, A. Nieto, FEBS Lett. 361, 255 (1995).
61. Z. Zhang et al. DNA Cell Biol. 16 (1), 73 (1997).
62. B. C. Misra, E. S. Srivatan, Am J. Hum. Genet. 455, 65 (1989).
63. G. A. Lammie et al. Oncogene 6, 439 (1991).
64. G. A. Lammie, G. Peters, In Cancer Cells Vol. 3 (11), 413 (1991), Cold Spring Harbor Laboratory Press, Cold Spring Harbor, N.Y.
65. S. Brookes, et al. Genes Chromosomes & Cancer 4, 290 (1992).
66. R. A. Jesudasan, et al., Anticancer Res. 14, L1727 (1994).
67. G. M. Hampton, et al. Proc. Natl. Acad. Sci. USA. 91, 6953 (1994).
68. R. A. Jesudasan, et al. Am. J. Hum. Genet. 56, 705 (1995).
69 P. J. Saxon, E. S. Srivatan, E. J. Stanbridge, EMBO J. 5, 3461 (1986).
70. M. Koi, et al., Mol. Carcinogenesis. 2, 12 (1989).
71. I. Linnoila et al., Am. J. Clin. Path. 97, 235 (1992).
72. J. L. Broers et al. Lab. Invest. 66, 337 (1992).
73. A. Sandmoller et al., Cell Growth Differ. 6, 97 (1995).
74. F. J. DeMayo et al., Am. J. Physiol. 261, L70 (1991).
75. A. Weerartna et al. Clin. Cancer Res. 3, 2295 (1997).
76. A. B. Mukherjee, L. Murty, J. Y. Chou, Mol. Cell. Endocrinol. 94, R15 (1993).
77. H. P. Erickson, N. A. Carrell, J. Biol. Chem., 258 (23): 14539-44 (1983).
78. A. Morla, Z. Zhang, E. Ruoslahti, Nature, 13 (367): 193-6 (1994).
79. S. Arii, et al., M. Hum. Cell 12, 25-30 (1999).
80. M. E. Beckner, Cancer Invest. 17, 594-623 (1999).
81. V. T. Byzova et al., Mol. Cell 6, 851-860 (2000).
82. P. Carmeliet. and R. K. Jain, Nature 407, 249-257 (2000).
83. L. Chandrasekaran et al., Mol. Biol. Cell 11, 2885-2900 (2000).
84. G. Collo and M. S. Pepper, J. Cell Sci. 112, 569-578 (1999).
85. J. P. Crew, Eur. Urol. 35, 2-8 (1999).
86. M. M. Eatock et al., Cancer Treat. Rev. 26, 191-204 (2000).
87. N. Ferrara et al., Kidney Int. 56, 794-814 (1999a).
88. N. Ferrara and K. Alitalo, Nat. Med. 5, 1359-1364 (1999b).
89. C. Frelin et al., Ann. Endocrinol. (Paris) 61, 70-74 (2000).
90. P. Gerwins, et al., Crit. Rev. Oncol. Hematol. 34, 185-194 (2000).
91. M. B. Grant, et al., Diabetes 47, 1335-1340 (1998).
92. M. Hagedorn and A. Bikfalvi, Crit. Rev. Oncol. 34, 89-110 (2000).
92. S. Kim et al., J. Biol. Chem. 275, 33920-33928 (2000).
93. W. D. Klohs and J. M. Hamby, Curr. Opin. Biotechnol. 10, 544-549 (1999).
94. J. A. Maier et al., Cytokine. 11, 134-139 (1999).
95. G. McMahon, Oncologist 5 Suppl 1, 3-10 (2000).
96. A. B. Mukherjee et al., Cell Mol. Life Sci. 55, 771-787 (1999).
97. Ruoslahti, E., Adv. Cancer Res. 76, 1-20 (1999).
98. J. M. Schlaeppi and J. M. Wood, Cancer Metastasis Rev. 18, 473-481 (1999).
99. R. Soldi et al., EMBO J. 18, 882-892 (1999).
100. E. Szabo et al., Cell Growth Differ. 9:475-85.
101. H. Takei et al., Int. J. Oncol. 12, 517-523 (1998).
102. T. Veikkola et al., Cancer Res. 60, 203-212 (2000).
103. Z. Zhu and L. Witte Invest. New Drugs 17, 195-212 (1999).

1-184. (canceled)

185. A method of increasing the number of lymphoid white cells in the circulation of a mammal comprising administering rhUG.

186. The method of claim 185 in which the lymphoid white cells are T cells.

187. The method of claim 185 in which the lymphoid white cells are B cells.

188. The method of claim 185 in which the lymphoid white cells are dendritic cells.

189. The method of claim 185 in which the lymphocytes are NK cells.

190. A method of decreasing the number of polymorphonuclear (PMN) white cells in the circulation of a mammal comprising administering rhUG.

191. The method of claim 190 in which the PMN white cells are leukocytes.

192. The method of claim 191 in which the PMN white cells are neutrophils.

193. The method of claim 191 in which the PMN white cells are eosinophils.

194. The method of claim 191 in which the PMN white cells are monocytes.

195. The method of claim 191 in which the PMN white cells are macrophages.

196. A method of diverting hematopoietic stem cell differentiation in a mammal away from the myeloid lineage and towards the lymphoid lineage comprising administering uteroglobin.

197. A method of enhancing ATPase activity in a CD11b-positive cell in a mammal comprising administering rhUG.

198. The method of claim 197 in which the CD11b-positive cell is a white cell of myeloid lineage.

199. The method of claim 197 in which the CD11b-positive cell is a neutrophil.

200. The method of claim 197 in which the CD11b-positive cell is an eosinophil.

201. The method of claim 197 in which the CD11b-positive cell is a monocyte.

202. The method of claim 197 in which the CD11b-positive cell is a macrophage.

203. A method of inhibiting ATPase activity in a non-cancerous CD71-positive cell in a mammal comprising administering rhUG.
